(19) United States
(12) Patent Application Publication
(10) Pub. No.: US 2015/0144937 A1
(43) Pub. Date: May 28, 2015

(54) COMPOUND FOR ORGANIC OPTOELECTRONIC DEVICE, ORGANIC LIGHT EMITTING DIODE COMPRISING SAME, AND DISPLAY DEVICE COMPRISING ORGANIC LIGHT EMITTING DIODE

(71) Applicant: SAMSUNG SDI CO., LTD., Yongin-si (KR)

(72) Inventors: Moo-Jin PARK, Suwon-si (KR); Eun-Sun YU, Suwon-si (KR); Mi-Young CHAE, Suwon-si (KR); Byung-Ku KIM, Suwon-si (KR); Hyo-Ju SEO, Suwon-si (KR); Jae-Deuk YANG, Suwon-si (KR)

(21) Appl. No.: 14/612,988

(22) Filed: Feb. 3, 2015

Related U.S. Application Data
(63) Continuation of application No. PCT/KR2013/004655, filed on May 28, 2013.

(30) Foreign Application Priority Data
Oct. 8, 2012 (KR) 10-2012-0111342

(51) Int. Cl. H01L 51/00; C07D 495/10; C07D 471/10; C07D 491/107; C07D 401/14; C07F 7/08

(52) U.S. Cl. CPC H01L 51/0072; H01L 51/0067; H01L 51/0094; C07D 401/14; C07F 7/082; H01L 51/0073; C07D 471/10; C07D 491/107; C07D 495/10; H01L 51/5016
USPC 257/40; 544/212; 544/294; 546/18; 546/14; 544/229

(57) ABSTRACT

Provided are a compound represented by the following Chemical Formula 1 for an organic optoelectronic device, an organic light emitting diode including the same, and a display device including the organic light emitting diode. The structure of Chemical Formula 1 is described in the specification.

The compound for the organic photoelectric device provides an organic light emitting diode having excellent life-span characteristics due to excellent electrochemical and thermal stability and high luminous efficiency at a low driving voltage.

### CROSS-REFERENCE TO RELATED APPLICATIONS

This application is a continuation of pending International Application No. PCT/KR2013/004655, entitled “Compound for Organic Optoelectronic Device, Organic Light Emitting Diode Comprising Same, and Display Device Comprising Organic Light Emitting Diode,” which was filed on May 28, 2013, the entire contents of which are hereby incorporated by reference.

Korean Patent Application No. 10-2012-0111342, filed on Oct. 8, 2012, in the Korean Intellectual Property Office, and entitled: “Compound for Organic Optoelectronic Device, Organic Light Emitting Diode Comprising Same,” is incorporated by reference herein in its entirety.

### BACKGROUND

1. Field

A compound for an organic optoelectronic device having excellent life-span, efficiency, electrochemical stability, and thermal stability, an organic light emitting diode including the compound, and a display device including the organic light emitting diode are disclosed.

2. Description of the Related Art

An organic optoelectronic device is a device requiring a charge exchange between an electrode and an organic material by using holes or electrons.

An organic optoelectronic device may be classified as follows in accordance with its driving principles. A first organic optoelectronic device is an electronic device driven as follows: excitons are generated in an organic material layer by photons from an external light source; the excitons are separated into electrons and holes; and the electrons and holes are transferred to different electrodes as a current source (voltage source).

A second organic optoelectronic device is an electronic device driven as follows: a voltage or a current is applied to at least two electrodes to inject holes and/or electrons into an organic material semiconductor positioned at an interface of the electrodes, and the device is driven by the injected electrons and holes.

Examples of an organic optoelectronic device include an organic photoelectric device, an organic light emitting diode, an organic solar cell, an organic photo conductor drum, an organic transistor, and the like, which require a hole injecting or transport material, an electron injecting or transport material, or a light emitting material.

Particularly, an organic light emitting diode (OLED) has recently drawn attention due to an increase in demand for flat panel displays. In general, organic light emission refers to conversion of electrical energy into photo-energy.

Such an organic light emitting diode converts electrical energy into light by applying current to an organic light emitting material. It has a structure in which a functional organic material layer is interposed between an anode and a cathode. The organic material layer includes a multi-layer including different materials, for example a hole injection layer (HIL), a hole transport layer (HTL), an emission layer, an electron transport layer (ETL), and an electron injection layer (EIL), in order to improve efficiency and stability of an organic light emitting diode.

In such an organic light emitting diode, when a voltage is applied between an anode and a cathode, holes from the anode and electrons from the cathode are injected to an organic material layer and recombined to generate excitons having high energy. The generated excitons generate light having certain wavelengths while shifting to a ground state.

Recently, it has become known that a phosphorescent light emitting material may be used for a light emitting material of an organic light emitting diode in addition to the fluorescent light emitting material. Such a phosphorescent material emits lights by transporting the electrons from a ground state to an exited state, non-radiance transiting of a singlet exciton to a triplet exciton through intersystem crossing, and transiting a triplet exciton to a ground state to emit light.

As described above, in an organic light emitting diode, an organic material layer includes a light emitting material and a charge transport material, for example a hole injection material, a hole transport material, an electron transport material, an electron injection material, and the like.

The light emitting material is classified as blue, green, and red light emitting materials according to emitted colors, and yellow and orange light emitting materials to emit colors approaching natural colors.

When one material is used as a light emitting material, a maximum light emitting wavelength is shifted to a long wavelength or color purity decreases because of interactions between molecules, or device efficiency decreases because of a light emitting quenching effect. Therefore, a host/dopant system is included as a light emitting material in order to improve color purity and increase luminous efficiency and stability through energy transfer.

In order to implement excellent performance of an organic light emitting diode, a material constituting an organic material layer, for example a hole injection material, a hole transport material, a light emitting material, an electron transport material, an electron injection material, and a light emitting material such as a host and/or a dopant, should be stable and have good efficiency. However, development of an organic material layer forming material for an organic light emitting diode has thus far not been satisfactory and thus there is a need for a novel material. This material development is also required for other organic optoelectronic devices.

The low molecular organic light emitting diode is manufactured as a thin film in a vacuum deposition method and can have good efficiency and life-span performance. A polymer organic light emitting diode is manufactured in an inkjet or spin coating method has an advantage of low initial cost and being large-sized.

Both low molecular organic light emitting and polymer organic light emitting diodes have an advantage of self-light emitting, high speed response, wide viewing angle, ultra-thin, high image quality, durability, large driving temperature range, and the like. In particular, they have good visibility due to self-light emitting characteristics compared with a conventional LCD (liquid crystal display) and have an advantage of decreasing thickness and weight of LCD up to a third, because they do not need a backlight.

In addition, since they have a response speed 1000 time faster microsecond unit than LCD, they can realize a perfect motion picture without after-image. Based on these advantages, they have been remarkably developed to have 80 times efficiency and more than 100 times life-span since they come out for the first time in the late 1980s. Recently, they keep being rapidly larger such as a 40-inch organic light emitting diode panel.

They are simultaneously required to have improved luminous efficiency and life-span in order to be larger. Herein, their luminous efficiency need smooth combination between holes and electrons in an emission layer. However, since an organic material in general has slower electron mobility than hole mobility, it has a drawback of inefficient combination between holes and electrons. Accordingly, while increasing electron injection and mobility from a cathode and simultaneously preventing movement of holes is required.

In order to improve life-span, a material crystallization caused by Joule heats generated during device operating is required to be prevented. Accordingly, there has been a strong need for an organic compound having excellent electron injection and mobility, and high electrochemical stability.

### SUMMARY

A compound for an organic optoelectronic device that may act as hole injection and transport or electron injection and transport material, and also act as a light emitting host along with an appropriate dopant is provided.

An organic light emitting diode having excellent life-span, efficiency, driving voltage, electrochemical stability and thermal stability and a display device including the same are provided.

One embodiment of the present invention provides a compound for an organic optoelectronic device represented by the following Chemical Formula 1.

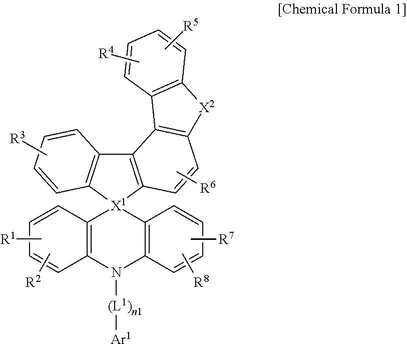

In the Chemical Formula 1, X¹ is C or Si, X² is O, S, SO₂ (O═S═O), PO (P═O), CR′R″ or NR′, wherein R′, R″ and R¹ to R⁸ are independently hydrogen, deuterium, a halogen, a cyano group, a hydroxyl group, an amino group, a substituted or unsubstituted C1 to C20 amine group, a nitro group, a carboxyl group, a ferrocenyl group, a substituted or unsubstituted C1 to C20 alkyl group, a substituted or unsubstituted C6 to C30 aryl group, a substituted or unsubstituted C2 to C30 heteroaryl group, a substituted or unsubstituted C1 to C20 alkoxy group, a substituted or unsubstituted C6 to C20 aryloxy group, a substituted or unsubstituted C3 to C40 silyloxy group, a substituted or unsubstituted C1 to C20 acyl group, a substituted or unsubstituted C2 to C20 alkoxycarbonyl group, a substituted or unsubstituted C2 to C20 acyloxy group, a substituted or unsubstituted C2 to C20 acylamino group, a substituted or unsubstituted C2 to C20 alkoxycarbonylamino group, a substituted or unsubstituted C7 to C20 aryloxycarbonylamino group, a substituted or unsubstituted C1 to C20 sulfamoylamino group, a substituted or unsubstituted C1 to C20 sulfonyl group, a substituted or unsubstituted C1 to C20 alkylthiol group, a substituted or unsubstituted C6 to C20 arylthiol group, a substituted or unsubstituted C1 to C20 heterocyclothiol group, a substituted or unsubstituted C1 to C20 ureide group, a substituted or unsubstituted C3 to C40 silyl group, or a combination thereof, L¹ is a substituted or unsubstituted C2 to C6 alkenylene group, a substituted or unsubstituted C2 to C6 alkynylene group, a substituted or unsubstituted C6 to C30 arylene group, a substituted or unsubstituted C2 to C30 heteroarylene group, or a combination thereof, n1 is an integer of 0 to 3, Ar¹ is a substituted or unsubstituted C6 to C30 aryl group or a substituted or unsubstituted C2 to C30 heteroaryl group, and at least one of Ar¹, R⁸ and R′ is a substituted or unsubstituted C2 to C30 heteroaryl group having electron characteristics.

The X² may be 0, S or NR′, and the Ar¹ may be a substituted or unsubstituted C2 to C30 heteroaryl group having electron characteristics.

The X² may be NR′, wherein R′ may be a substituted or unsubstituted C6 to C30 aryl group.

The X² may be NR′, wherein R′ may be a substituted or unsubstituted C2 to C30 heteroaryl group having electron characteristics.

The Ar¹ may be a substituted or unsubstituted C6 to C30 aryl group.

The compound represented by the Chemical Formula 1 may be represented by the following Chemical Formula 2.

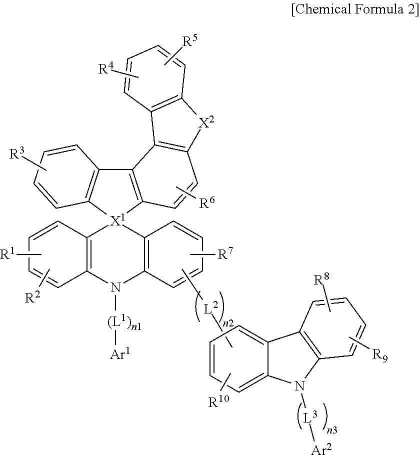

In the Chemical Formula 2, X′ is C or Si, X² is O, S, SO₂ (O═S═O), PO (P═O), CR′R″ or NR′, wherein R′, R″ and R¹ to R¹⁰ are independently hydrogen, deuterium, a halogen, a cyano group, a hydroxyl group, an amino group, a substituted or unsubstituted C1 to C20 amine group, a nitro group, a carboxyl group, a ferrocenyl group, a substituted or unsubstituted C1 to C20 alkyl group, a substituted or unsubstituted C6 to C30 aryl group, a substituted or unsubstituted C2 to C30 heteroaryl group, a substituted or unsubstituted C1 to C20 alkoxy group, a substituted or unsubstituted C6 to C20 aryloxy group, a substituted or unsubstituted C3 to C40 silyloxy group, a substituted or unsubstituted C1 to C20 acyl group, a substituted or unsubstituted C2 to C20 alkoxycarbonyl group, a substituted or unsubstituted C2 to C20 acyloxy group, a substituted or unsubstituted C2 to C20 acylamino group, a substituted or unsubstituted C2 to C20 alkoxycarbonylamino group, a substituted or unsubstituted C7 to C20 aryloxycarbonylamino group, a substituted or unsubstituted C1 to C20 sulfamoylamino group, a substituted or unsubstituted C1 to C20 sulfonyl group, a substituted or unsubstituted C1 to C20 alkylthiol group, a substituted or unsubstituted C6 to C20 arylthiol group, a substituted or unsubstituted C1 to C20 heterocyclothiol group, a substituted or unsubstituted C1 to C20 ureide group, a substituted or unsubstituted C3 to C40 silyl group, or a combination thereof, L¹ to L³ are independently substituted or unsubstituted C2 to C6 alkenylene group, a substituted or unsubstituted C2 to C6 alkynylene group, a substituted or unsubstituted C6 to C30 arylene group, a substituted or unsubstituted C2 to C30 heteroarylene group, or a combination thereof, n1 to n3 are independently integers of 0 to 3, Ar¹ and Ar² are independently a substituted or unsubstituted C6 to C30 aryl group or a substituted or unsubstituted C2 to C30 heteroaryl group, and at least one of Ar¹, Ar², R⁵, R⁸ and R′ is a substituted or unsubstituted C2 to C30 heteroaryl group having electron characteristics.

The X² may be O, S or CR′R″, and the Ar¹ may be a substituted or unsubstituted C2 to C30 heteroaryl group having electron characteristics.

The X² may be O or S, and the Ar² may be a substituted or unsubstituted C6 to C30 aryl group.

The X² may be O or S, the Ar² may be a substituted or unsubstituted C2 to C30 heteroaryl group having electron characteristics, and the Ar¹ may be substituted or unsubstituted C6 to C30 aryl group.

The X¹ may be C.

The substituted or unsubstituted C2 to C30 heteroaryl group having electron characteristics may be represented by one of the following Chemical Formulae 3 to 7.

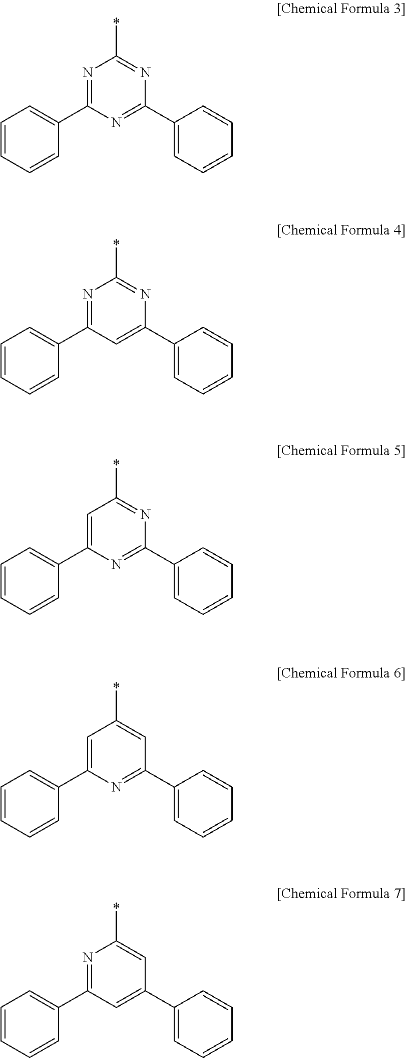 [SMILES: *c1nc(-c2ccccc2)nc(-c2ccccc2)n1 ; *c1nc(-c2ccccc2)cc(-c2ccccc2)n1 ; *c1cc(-c2ccccc2)nc(-c2ccccc2)n1 ; *c1cc(-c2ccccc2)nc(-c2ccccc2)c1 ; *c1cc(-c2ccccc2)cc(-c2ccccc2)n1]

The Ar¹ and Ar² are independently, a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenylyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, or a combination thereof.

L¹ to L³ are independently substituted or unsubstituted phenylene group, a substituted or unsubstituted biphenylene group, a substituted or unsubstituted terphenylene group, a substituted or unsubstituted naphthylene group, a substituted or unsubstituted anthracenylene group, a substituted or unsubstituted phenanthrylene group, a substituted or unsubstituted pyrenylene group, a substituted or unsubstituted fluorenylene group, a substituted or unsubstituted naphthacenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group and a substituted or unsubstituted phenoxazinyl group.

At least one of the R¹ to R¹⁰ may be a substituted or unsubstituted C3 to C40 silyl group.

At least one of the R¹ to R¹⁰ may be a substituted C3 to C40 silyl group, wherein at least one hydrogen of the substituted silyl group may be substituted with a C1 to C10 alkyl group or a C6 to C15 aryl group.

The compound for an organic optoelectronic device may have triplet exciton energy (T1) of 2.0 eV or greater.

In another embodiment of the present invention, an organic light emitting diode includes an anode, and at least one or more organic thin layer between the anode and the cathode, wherein at least one of the organic thin layers includes the compound for an organic optoelectronic device.

The organic thin layer may be selected from the group consisting of an emission layer, a hole transport layer (HTL), a hole injection layer (HIL), an electron transport layer (ETL), an electron injection layer (EIL), a hole blocking layer and a combination thereof.

The compound for an organic optoelectronic device may be included in an emission layer.

In yet another embodiment of the present invention, a display device includes the organic light emitting diode according to the embodiment of the present invention.

A compound having high hole or electron transport properties, film stability thermal stability and high triplet exciton energy is provided.

Such a compound may be used as a hole injection/transport material, host material, or an electron injection/transport material of an emission layer. The organic optoelectronic device using the same has improved life-span characteristics due to excellent electrochemical and thermal stability, and high luminous efficiency at a low driving voltage.

### BRIEF DESCRIPTION OF THE DRAWINGS

Features will be apparent to those of skill in the art by describing in detail exemplary embodiments with reference to the attached drawings in which:

FIGS. 1 to 5 illustrate cross-sectional views showing organic light emitting diodes according to various embodiments of the present invention using a compound for an organic optoelectronic device according to one embodiment of the present invention.

### DESCRIPTION OF REFERENCE NUMERALS INDICATING PRIMARY ELEMENTS IN THE DRAWINGS

100: organic light emitting diode
110: cathode
120: anode
105: organic thin layer
130: emission layer
140: hole transport layer (HTL)
150: electron transport layer (ETL)
160: electron injection layer (EIL)
170: hole injection layer (HIL)
230: emission layer+electron transport layer (ETL)

### DETAILED DESCRIPTION

Hereinafter, embodiments of the present invention are described in detail. However, these embodiments are exemplary, and do not limit the present invention, and the present invention is defined by the scope of the claims which will be described later.

In the present specification, when specific definition is not otherwise provided, “substituted” refers to one substituted with deuterium, a halogen, hydroxy group, an amino group, a substituted or unsubstituted C1 to C30 amine group, a nitro group, a substituted or unsubstituted C3 to C40 silyl group, a C1 to C30 alkyl group, a C1 to C10 alkylsilyl group, a C3 to C30 cycloalkyl group, C6 to C30 aryl group, a C1 to C20 alkoxy group, a fluoro group, a C1 to C10 trifluoroalkyl group such as trifluoromethyl group and the like or a cyano group instead of at least one hydrogen of a substitutent or a compound.

Two substitutents of the substituted halogen, hydroxy group, amino group, substituted or unsubstituted C1 to C20 amine group, nitro group, substituted or unsubstituted C3 to C40 silyl group, C1 to C30 alkyl group, C1 to C10 alkylsilyl group, C3 to C30 cycloalkyl group, C6 to C30 aryl group, C1 to C20 alkoxy group, fluoro group, C1 to C10 trifluoroalkyl group such as trifluoromethyl group and the like or cyano group may be fused with each other to form a ring.

In the present specification, when specific definition is not otherwise provided, “hetero” refers to one including 1 to 3 hetero atoms selected from the group consisting of N, O, S, and P, and remaining carbons in one compound or substituent.

In the present specification, when a definition is not otherwise provided, the term “combination thereof” refers to at least two substituents bound to each other by a linker, or at least two substituents condensed to each other.

In the present specification, when a definition is not otherwise provided, “alkyl group” refers to an aliphatic hydrocarbon group. The alkyl group may be “a saturated alkyl group” without a double bond or a triple bond.

The alkyl group may be a branched, linear, or cyclic alkyl group.

The “alkenyl group” refers to a functional group consisting of at least one carbon-carbon double bond of at least two carbons, and the “alkynylene group” refers to a functional group consisting of at least one carbon-carbon triple bond of at least two carbons.

The alkyl group may be a C1 to C20 alkyl group. More specifically, the alkyl group may be a C1 to C10 alkyl group or a C1 to C6 alkyl group.

For example, a C1 to C4 alkyl group may have 1 to 4 carbon atoms and may be selected from the group consisting of methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

“Aromatic group” refers to a cyclic functional group where all elements have p-orbitals, and these p-orbitals forms conjugation. Specific examples are aryl group and a heteroaryl group.

“Aryl group” refers to a substituent including all element of the cycle having p-orbitals which form conjugation, and may be monocyclic, polycyclic or fused ring polycyclic (i.e., rings sharing adjacent pairs of carbon atoms) functional group.

“Heteroaryl group” refers to an aryl group including 1 to 3 hetero atoms selected from the group consisting of N, O, S, P, and Si and remaining carbons. The heteroaryl group may be a fused ring where each ring may include the 1 to 3 heteroatoms.

In the present specification, the carbazole-based derivative may refer to a substituted structure where a nitrogen atom of a substituted or unsubstituted carbazolyl group is substituted with a hetero atom except nitrogen, or carbon. Specific examples may be dibenzofuran (dibenzofuranyl group), dibenzothiophene (dibenzothiophenyl group), fluorene (fluorenyl group), and the like. For specific examples, the heteroatom may include —O—, —S—, —S(O)—, —S(O)₂— or —NR′—.

In the present specification, hole characteristics refer to characteristics that holes formed in the anode is easily injected into the emission layer and transported in the emission layer due to conductive characteristics according to HOMO level. More specifically, it is similar to electron-repelling characteristics.

Electron characteristics refer to characteristics that electron formed in the cathode is easily injected into the emission layer and transported in the emission layer due to conductive characteristics according to LUMO level. More specifically, it is similar to electron-withdrawing characteristics.

A compound for an organic optoelectronic device according to one embodiment of the present invention has a structure including a fused ring core and optionally various substituents.

The core structure may be used as a light emitting material, a hole injection material or a hole transport material of an organic optoelectronic device. Particularly, it may be suitable as a hole injection material or a hole transport material.

The compound for an organic optoelectronic device includes a core part and various substituents for a substitutent for substituting the core part and thus may have various energy bandgaps.

The compound may have an appropriate energy level depending on the substituents and thus, may fortify hole transport capability or electron transport capability of an organic optoelectronic device and bring about excellent effects on efficiency and driving voltage and also, have excellent electrochemical and thermal stability and thus, improve life-span characteristics during the operation of the organic optoelectronic device.

According to one embodiment of the present invention, the compound for an organic optoelectronic device is represented by the following Chemical Formula 1.

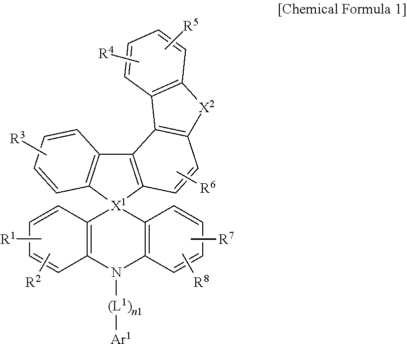

In the Chemical Formula 1, X′ is C or Si, X² is O, S, SO₂ (O═S═O), PO (P═O), CR′R″ or NR′, wherein R′, R″ and R¹ to R⁷ are independently hydrogen, deuterium, a halogen, a cyano group, a hydroxyl group, an amino group, a substituted or unsubstituted C1 to C20 amine group, a nitro group, a carboxyl group, a ferrocenyl group, a substituted or unsubstituted C1 to C20 alkyl group, a substituted or unsubstituted C6 to C30 aryl group, a substituted or unsubstituted C2 to C30 heteroaryl group, a substituted or unsubstituted C1 to C20 alkoxy group, a substituted or unsubstituted C6 to C20 aryloxy group, a substituted or unsubstituted C3 to C40 silyloxy group, a substituted or unsubstituted C1 to C20 acyl group, a substituted or unsubstituted C2 to C20 alkoxycarbonyl group, a substituted or unsubstituted C2 to C20 acyloxy group, a substituted or unsubstituted C2 to C20 acylamino group, a substituted or unsubstituted C2 to C20 alkoxycarbonylamino group, a substituted or unsubstituted C7 to C20 aryloxycarbonylamino group, a substituted or unsubstituted C1 to C20 sulfamoylamino group, a substituted or unsubstituted C1 to C20 sulfonyl group, a substituted or unsubstituted C1 to C20 alkylthiol group, a substituted or unsubstituted C6 to C20 arylthiol group, a substituted or unsubstituted C1 to C20 heterocyclothiol group, a substituted or unsubstituted C1 to C20 ureide group, a substituted or unsubstituted C3 to C40 silyl group, or a combination thereof, L¹ is a substituted or unsubstituted C2 to C6 alkenylene group, a substituted or unsubstituted C2 to C6 alkynylene group, a substituted or unsubstituted C6 to C30 arylene group, a substituted or unsubstituted C2 to C30 heteroarylene group, or a combination thereof, n1 is an integer of 0 to 3, Ar¹ is a substituted or unsubstituted C6 to C30 aryl group or a substituted or unsubstituted C2 to C30 heteroaryl group, and at least one of Ar¹, R⁸ and R′ is a substituted or unsubstituted C2 to C30 heteroaryl group having electron characteristics.

The structure of the above Chemical Formula 1 has a bipolar structure, and thereby insufficient electron transport (or injection) characteristics increase improving efficiency of a device.

More specifically, the X² may be O, S or NR′, and the Ar¹ may be a substituted or unsubstituted C2 to C30 heteroaryl group having electron characteristics. That is to say, when the substituent having electron characteristics is positioned at Ar¹, distributions of electrons and holes are separated, efficient hole and/or charge transfers are expected, and thereby efficiency of a device is improved.

More specifically, the X² may be NR′, wherein R′ is a substituted or unsubstituted C6 to C30 aryl group.

More specifically, the X² may be NR′, wherein R′ is a substituted or unsubstituted C2 to C30 heteroaryl group having electron characteristics, and the Ar¹ may be a substituted or unsubstituted C6 to C30 aryl group. That is to say, when the substituent having electron characteristics is positioned at R′ of NR′, distributions of electrons and holes are separated, efficient hole and/or charge transfers are expected, and thereby efficiency of a device is improved.

More specifically, the compound represented by the Chemical Formula 1 may be represented by the following Chemical Formula 2.

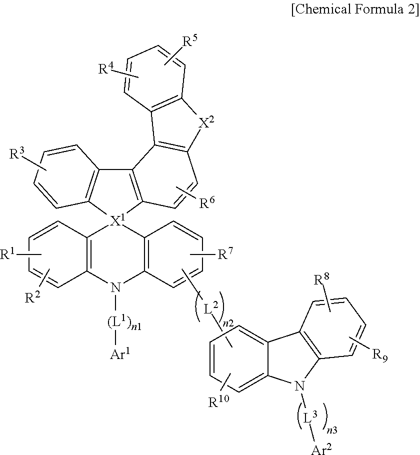

In the Chemical Formula 2, X¹ is C or Si, X² is O, S, SO₂ (O═S═O), PO (P═O), CR′R″ or NR′, wherein R′, R″ and R¹ to R¹⁰ are independently hydrogen, deuterium, a halogen, a cyano group, a hydroxyl group, an amino group, a substituted or unsubstituted C1 to C20 amine group, a nitro group, a carboxyl group, a ferrocenyl group, a substituted or unsubstituted C1 to C20 alkyl group, a substituted or unsubstituted C6 to C30 aryl group, a substituted or unsubstituted C2 to C30 heteroaryl group, a substituted or unsubstituted C1 to C20 alkoxy group, a substituted or unsubstituted C6 to C20 aryloxy group, a substituted or unsubstituted C3 to C40 silyloxy group, a substituted or unsubstituted C1 to C20 acyl group, a substituted or unsubstituted C2 to C20 alkoxycarbonyl group, a substituted or unsubstituted C2 to C20 acyloxy group, a substituted or unsubstituted C2 to C20 acylamino group, a substituted or unsubstituted C2 to C20 alkoxycarbonylamino group, a substituted or unsubstituted C7 to C20 aryloxycarbonylamino group, a substituted or unsubstituted C1 to C20 sulfamoylamino group, a substituted or unsubstituted C1 to C20 sulfonyl group, a substituted or unsubstituted C1 to C20 alkylthiol group, a substituted or unsubstituted C6 to C20 arylthiol group, a substituted or unsubstituted C1 to C20 heterocyclothiol group, a substituted or unsubstituted C1 to C20 ureide group, a substituted or unsubstituted C3 to C40 silyl group, or a combination thereof, L¹ to L³ are independently substituted or unsubstituted C2 to C6 alkenylene group, a substituted or unsubstituted C2 to C6 alkynylene group, a substituted or unsubstituted C6 to C30 arylene group, a substituted or unsubstituted C2 to C30 heteroarylene group, or a combination thereof, n1 to n3 are independently integers of 0 to 3, Ar¹ and Ar² are independently a substituted or unsubstituted C6 to C30 aryl group or a substituted or unsubstituted C2 to C30 heteroaryl group, and at least one of Ar¹, Ar², R⁵, R⁸ and R′ is a substituted or unsubstituted C2 to C30 heteroaryl group having electron characteristics.

When the separate carbazolyl group is included like the Chemical Formula 2, various substituents may be introduced in the compound, and transfer paths of electrons and holes are separated to improve efficiency of a device.

The X² may be O, S or CR′R″, and the Ar¹ may be a substituted or unsubstituted C2 to C30 heteroaryl group having electron characteristics. That is to say, when the substituent having electron characteristics is positioned at Ar¹, transfer paths of electrons and holes are separated and thus efficiency of a device may be improved.

In one embodiment of the present invention, the X² may be O or S, and the Ar² may be a substituted or unsubstituted C6 to C30 aryl group.

In one embodiment of the present invention, the X² may be O or S, the Ar² may be a substituted or unsubstituted C2 to C30 heteroaryl group having electron characteristics, and the Ar¹ may be a substituted or unsubstituted C6 to C30 aryl group. That is to say, when the substituent having electron characteristics is positioned at Ar², transfer paths of electrons and holes are separated and thus efficiency of a device may be improved.

The X¹ may be C, but is not limited thereto.

The substituted or unsubstituted C2 to C30 heteroaryl group having electron characteristics may be a substituent represented by one of the following Chemical Formulae 3 to 7, but is not limited thereto.

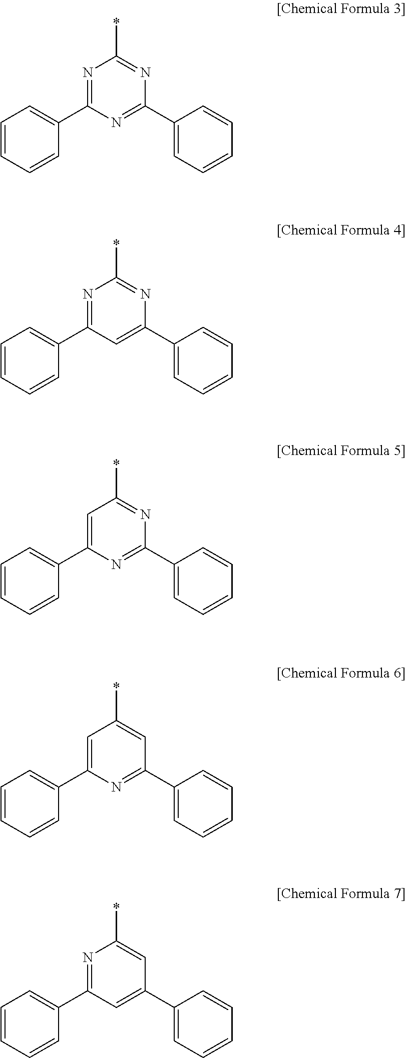 [SMILES: *c1nc(-c2ccccc2)nc(-c2ccccc2)n1 ; *c1nc(-c2ccccc2)cc(-c2ccccc2)n1 ; *c1cc(-c2ccccc2)nc(-c2ccccc2)n1 ; *c1cc(-c2ccccc2)nc(-c2ccccc2)c1 ; *c1cc(-c2ccccc2)cc(-c2ccccc2)n1]

The compound has a relatively large molecular weight and thus, may be suppressed from decomposition during the deposition.

The L¹ to L³ may be selectively adjusted to determine an entire conjugation length of the compound, and a triplet energy bandgap of the compound may be adjusted therefrom. Accordingly, characteristics of a material required of an organic photoelectric device may be realized. In addition, the triplet energy bandgap may also be adjusted by changing a bonding position of ortho, para, and meta.

Specific examples of the L¹ to L³ may be a substituted or unsubstituted phenylene group, a substituted or unsubstituted biphenylene group, a substituted or unsubstituted terphenylene group, a substituted or unsubstituted naphthylene group, a substituted or unsubstituted anthracenylene group, a substituted or unsubstituted phenanthrylene group, a substituted or unsubstituted pyrenylene group, a substituted or unsubstituted fluorenylene group, a substituted or unsubstituted naphthacenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group and a substituted or unsubstituted phenoxazinyl group.

In addition, the compound has steric hindrance and thus, small interaction among molecules and resultantly, may be suppressed from crystallization. Thereby, a yield of manufacturing a device may be improved. In addition, life-span characteristics of the device may be improved.

Furthermore, the compound has a relatively large molecular weight and thus, may be suppressed from decomposition during the deposition.

The Ar¹ and Ar² may be independently, a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenylyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, or a combination thereof, but are not limited thereto.

More specifically, at least one of the Ar¹ and Ar² may be a substituted or unsubstituted biphenyl group.

Or, at least one of the Ar¹ and Ar² may be a substituted or unsubstituted fluorenyl group.

At least one of the R¹ to R¹⁰ may be a substituted or unsubstituted C3 to C40 silyl group.

The silyl group reduces a deposit temperature during a manufacture of an organic optoelectronic device, and increases solubility for a solvent, and thus a manufacturing process of the device may be converted into a solution process.

More specifically, at least one of the R¹ to R¹⁰ may be a substituted C3 to C40 silyl group, wherein at least one hydrogen of the substituted silyl group may be substituted with a C1 to C10 alkyl group or a C6 to C15 aryl group.

Specific examples of the substituted silyl group may be a trimethylsilyl group, a triphenylsilyl group, and the like.

Specific examples of the compound for an organic optoelectronic device are as follows, but are not limited thereto.

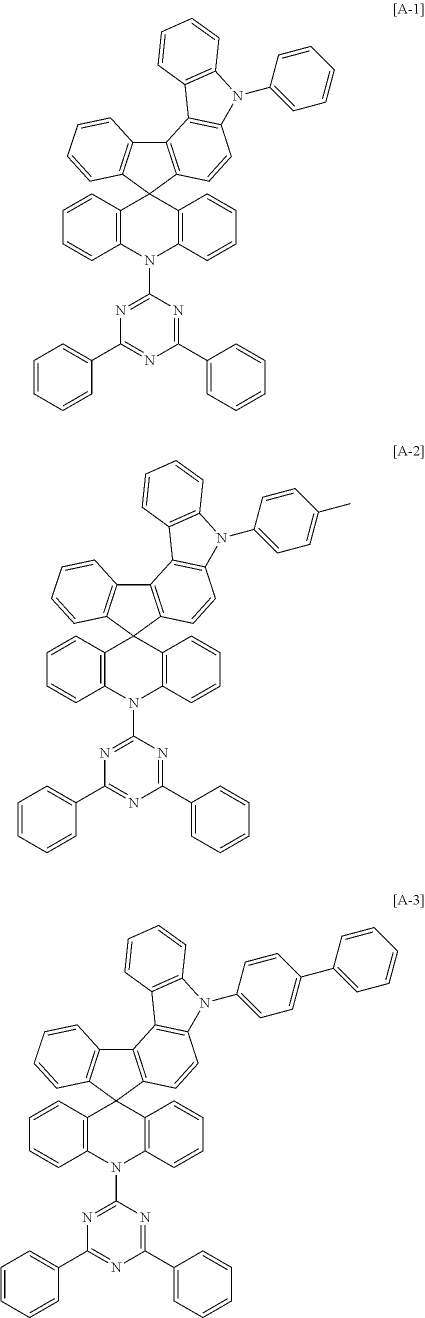 [SMILES: c1ccc(-c2nc(-c3ccccc3)nc(N3c4ccccc4C4(c5ccccc5-c5c4ccc4c5c5ccccc5n4-c4ccccc4)c4ccccc43)n2)cc1 ; Cc1ccc(-n2c3ccccc3c3c4c(ccc32)C2(c3ccccc3-4)c3ccccc3N(c3nc(-c4ccccc4)nc(-c4ccccc4)n3)c3ccccc32)cc1 ; c1ccc(-c2ccc(-n3c4ccccc4c4c5c(ccc43)C3(c4ccccc4-5)c4ccccc4N(c4nc(-c5ccccc5)nc(-c5ccccc5)n4)c4ccccc43)cc2)cc1]

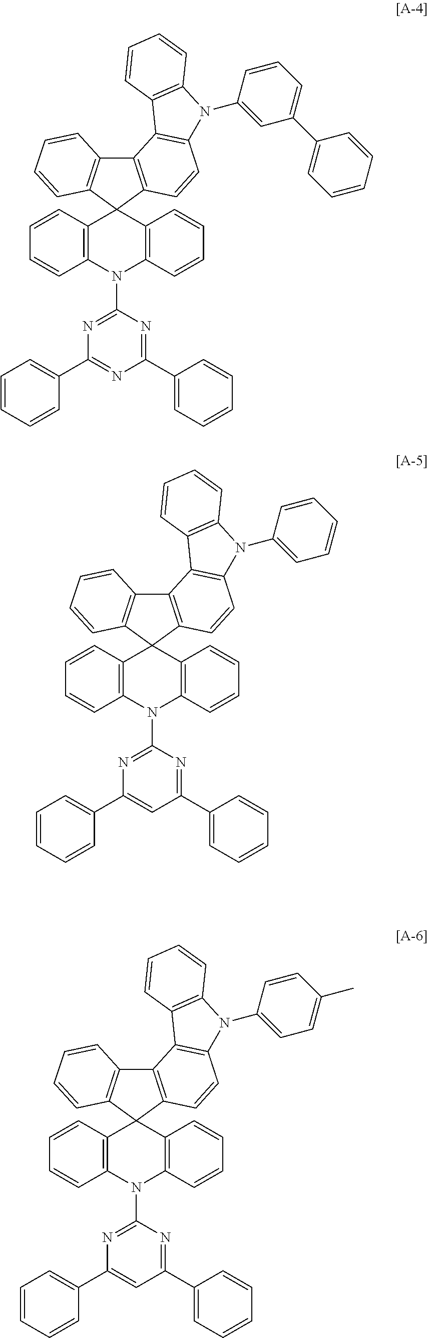

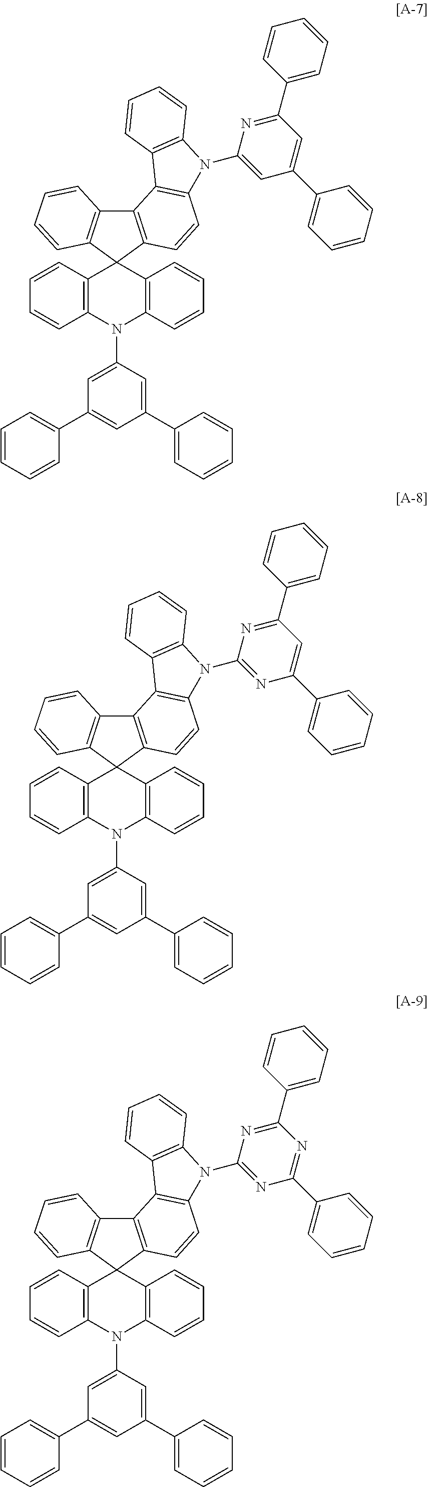 [SMILES: c1ccc(-c2cc(-c3ccccc3)cc(N3c4ccccc4C4(c5ccccc5-c5c4ccc4c5c5ccccc5n4-c4cc(-c5ccccc5)cc(-c5ccccc5)n4)c4ccccc43)c2)cc1 ; c1ccc(-c2cc(-c3ccccc3)cc(N3c4ccccc4C4(c5ccccc5-c5c4ccc4c5c5ccccc5n4-c4nc(-c5ccccc5)cc(-c5ccccc5)n4)c4ccccc43)c2)cc1 ; c1ccc(-c2cc(-c3ccccc3)cc(N3c4ccccc4C4(c5ccccc5-c5c4ccc4c5c5ccccc5n4-c4nc(-c5ccccc5)nc(-c5ccccc5)n4)c4ccccc43)c2)cc1]

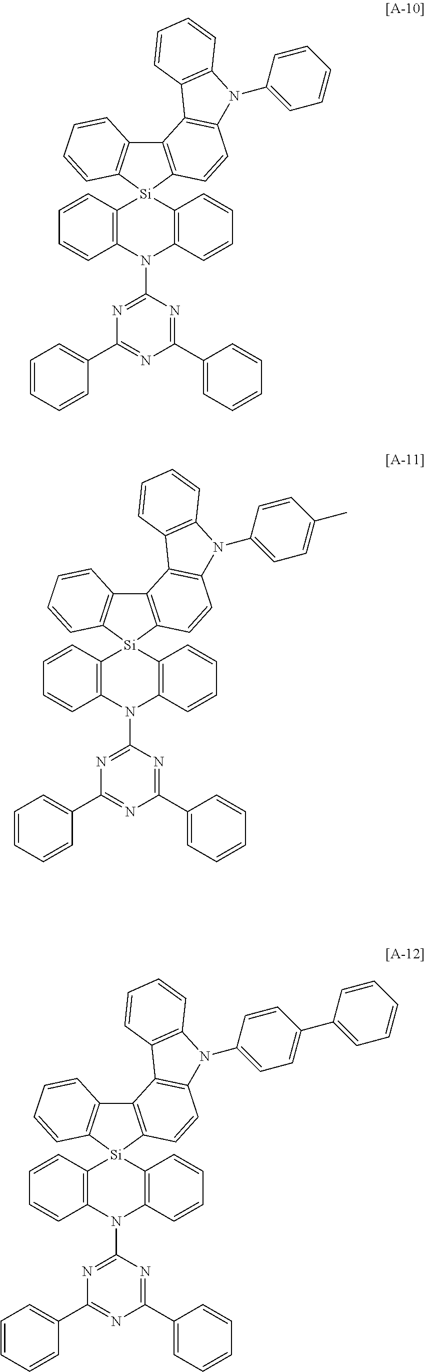 [SMILES: c1ccc(-c2nc(-c3ccccc3)nc(N3c4ccccc4[Si]4(c5ccccc5-c5c4ccc4c5c5ccccc5n4-c4ccccc4)c4ccccc43)n2)cc1 ; Cc1ccc(-n2c3ccccc3c3c4c(ccc32)[Si]2(c3ccccc3-4)c3ccccc3N(c3nc(-c4ccccc4)nc(-c4ccccc4)n3)c3ccccc32)cc1 ; c1ccc(-c2ccc(-n3c4ccccc4c4c5c(ccc43)[Si]3(c4ccccc4-5)c4ccccc4N(c4nc(-c5ccccc5)nc(-c5ccccc5)n4)c4ccccc43)cc2)cc1]

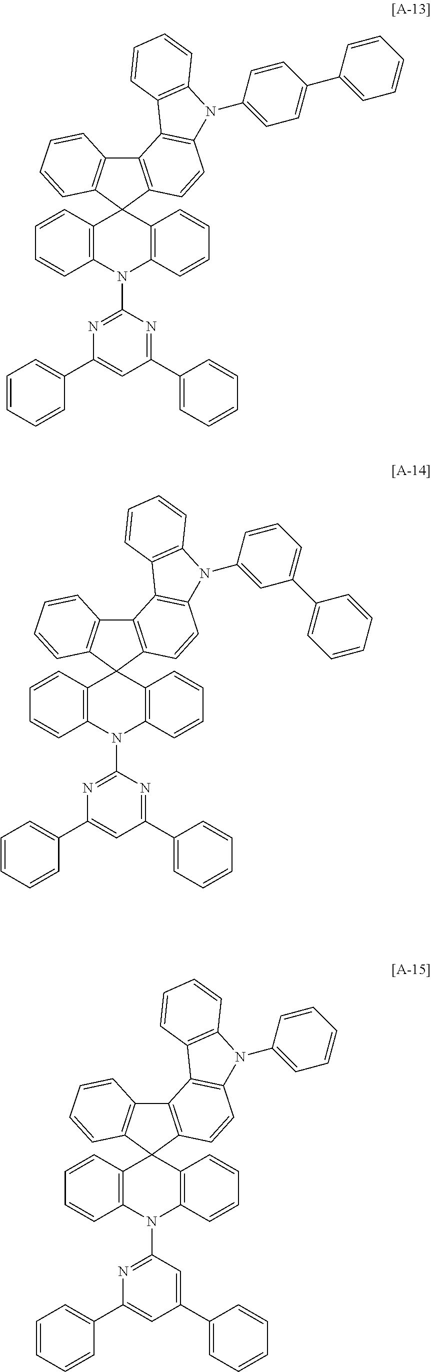 [SMILES: c1ccc(-c2ccc(-n3c4ccccc4c4c5c(ccc43)C3(c4ccccc4-5)c4ccccc4N(c4nc(-c5ccccc5)cc(-c5ccccc5)n4)c4ccccc43)cc2)cc1 ; c1ccc(-c2cccc(-n3c4ccccc4c4c5c(ccc43)C3(c4ccccc4-5)c4ccccc4N(c4nc(-c5ccccc5)cc(-c5ccccc5)n4)c4ccccc43)c2)cc1 ; c1ccc(-c2cc(-c3ccccc3)nc(N3c4ccccc4C4(c5ccccc5-c5c4ccc4c5c5ccccc5n4-c4ccccc4)c4ccccc43)c2)cc1]

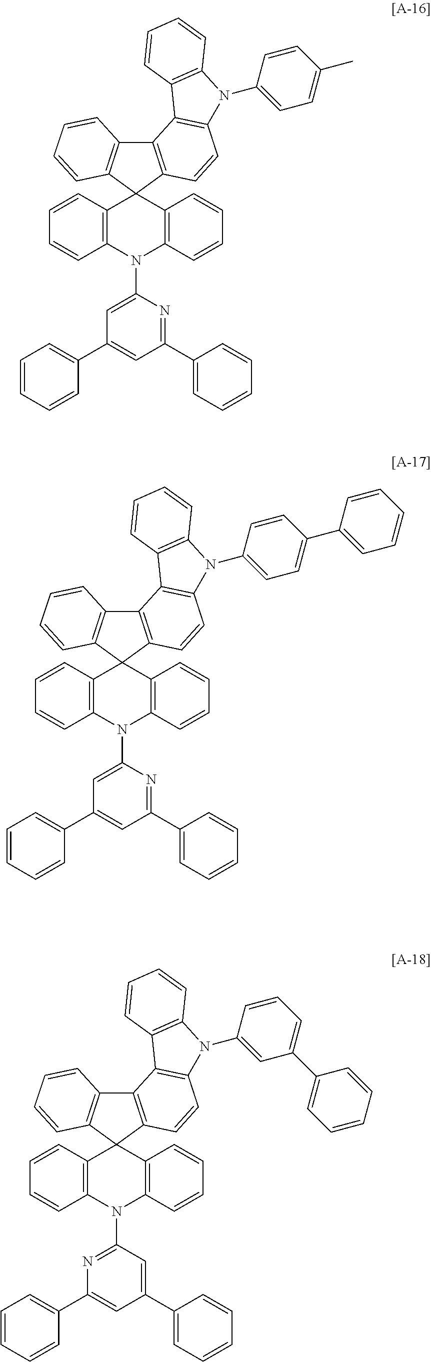 [SMILES: Cc1ccc(-n2c3ccccc3c3c4c(ccc32)C2(c3ccccc3-4)c3ccccc3N(c3cc(-c4ccccc4)cc(-c4ccccc4)n3)c3ccccc32)cc1 ; c1ccc(-c2ccc(-n3c4ccccc4c4c5c(ccc43)C3(c4ccccc4-5)c4ccccc4N(c4cc(-c5ccccc5)cc(-c5ccccc5)n4)c4ccccc43)cc2)cc1 ; c1ccc(-c2cccc(-n3c4ccccc4c4c5c(ccc43)C3(c4ccccc4-5)c4ccccc4N(c4cc(-c5ccccc5)cc(-c5ccccc5)n4)c4ccccc43)c2)cc1]

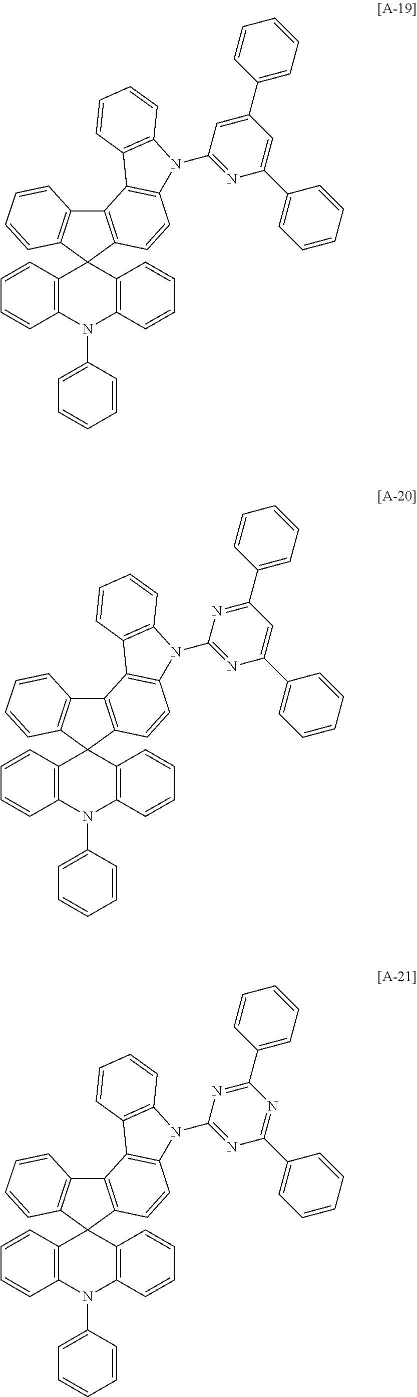 [SMILES: c1ccc(-c2cc(-c3ccccc3)nc(-n3c4ccccc4c4c5c(ccc43)C3(c4ccccc4-5)c4ccccc4N(c4ccccc4)c4ccccc43)c2)cc1 ; c1ccc(-c2cc(-c3ccccc3)nc(-n3c4ccccc4c4c5c(ccc43)C3(c4ccccc4-5)c4ccccc4N(c4ccccc4)c4ccccc43)n2)cc1 ; c1ccc(-c2nc(-c3ccccc3)nc(-n3c4ccccc4c4c5c(ccc43)C3(c4ccccc4-5)c4ccccc4N(c4ccccc4)c4ccccc43)n2)cc1]

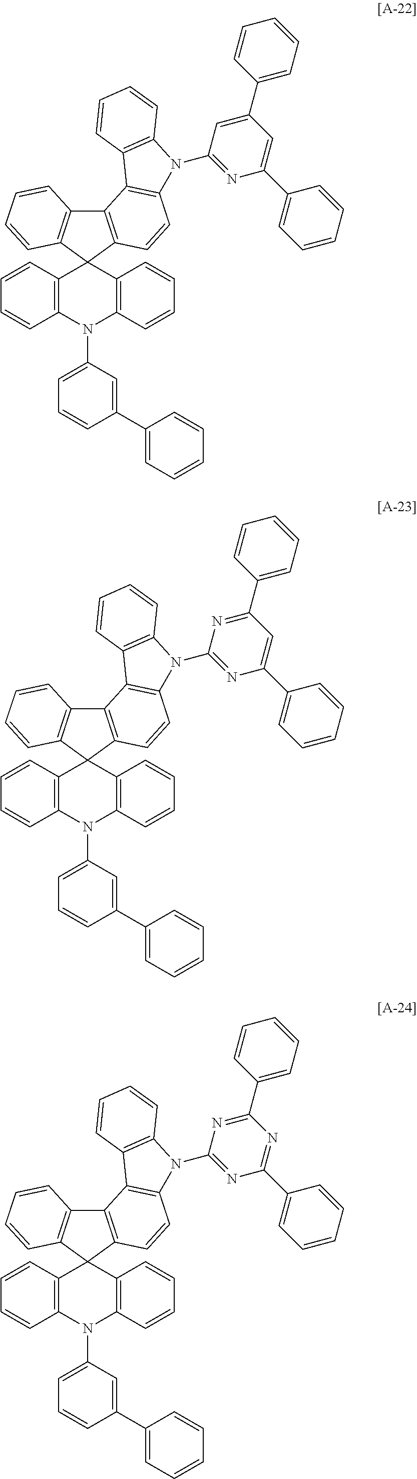 [SMILES: c1ccc(-c2cccc(N3c4ccccc4C4(c5ccccc5-c5c4ccc4c5c5ccccc5n4-c4cc(-c5ccccc5)cc(-c5ccccc5)n4)c4ccccc43)c2)cc1 ; c1ccc(-c2cccc(N3c4ccccc4C4(c5ccccc5-c5c4ccc4c5c5ccccc5n4-c4nc(-c5ccccc5)cc(-c5ccccc5)n4)c4ccccc43)c2)cc1 ; c1ccc(-c2cccc(N3c4ccccc4C4(c5ccccc5-c5c4ccc4c5c5ccccc5n4-c4nc(-c5ccccc5)nc(-c5ccccc5)n4)c4ccccc43)c2)cc1]

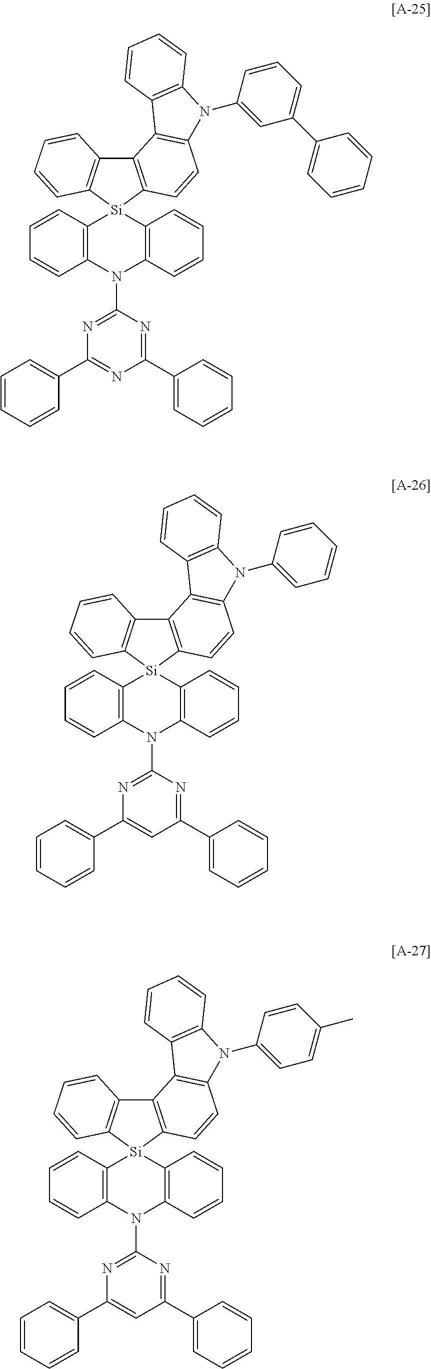 [SMILES: c1ccc(-c2cccc(-n3c4ccccc4c4c5c(ccc43)[Si]3(c4ccccc4-5)c4ccccc4N(c4nc(-c5ccccc5)nc(-c5ccccc5)n4)c4ccccc43)c2)cc1 ; c1ccc(-c2cc(-c3ccccc3)nc(N3c4ccccc4[Si]4(c5ccccc5-c5c4ccc4c5c5ccccc5n4-c4ccccc4)c4ccccc43)n2)cc1 ; Cc1ccc(-n2c3ccccc3c3c4c(ccc32)[Si]2(c3ccccc3-4)c3ccccc3N(c3nc(-c4ccccc4)cc(-c4ccccc4)n3)c3ccccc32)cc1]

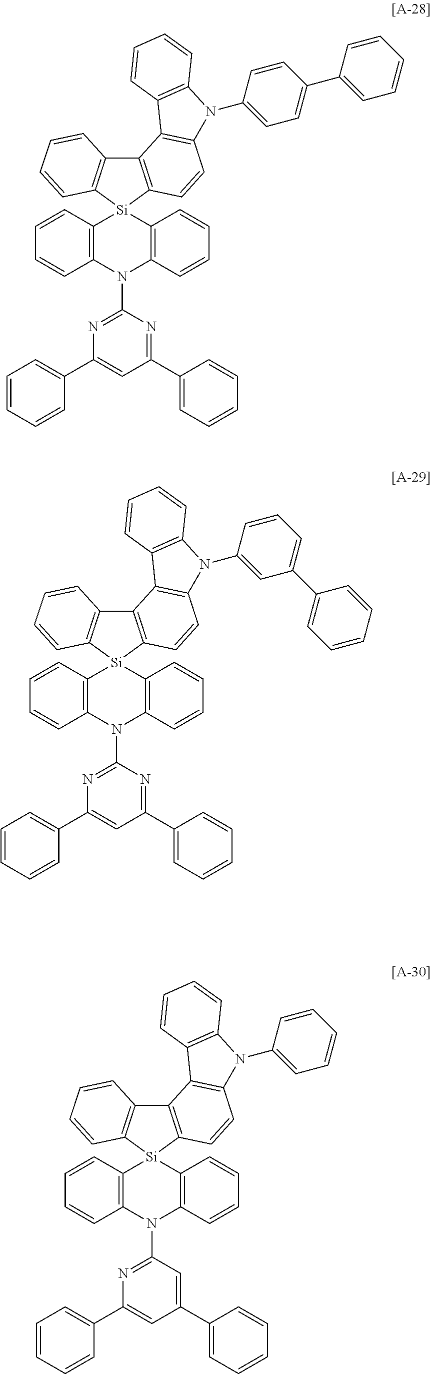 [SMILES: c1ccc(-c2ccc(-n3c4ccccc4c4c5c(ccc43)[Si]3(c4ccccc4-5)c4ccccc4N(c4nc(-c5ccccc5)cc(-c5ccccc5)n4)c4ccccc43)cc2)cc1 ; c1ccc(-c2cccc(-n3c4ccccc4c4c5c(ccc43)[Si]3(c4ccccc4-5)c4ccccc4N(c4nc(-c5ccccc5)cc(-c5ccccc5)n4)c4ccccc43)c2)cc1 ; c1ccc(-c2cc(-c3ccccc3)nc(N3c4ccccc4[Si]4(c5ccccc5-c5c4ccc4c5c5ccccc5n4-c4ccccc4)c4ccccc43)c2)cc1]

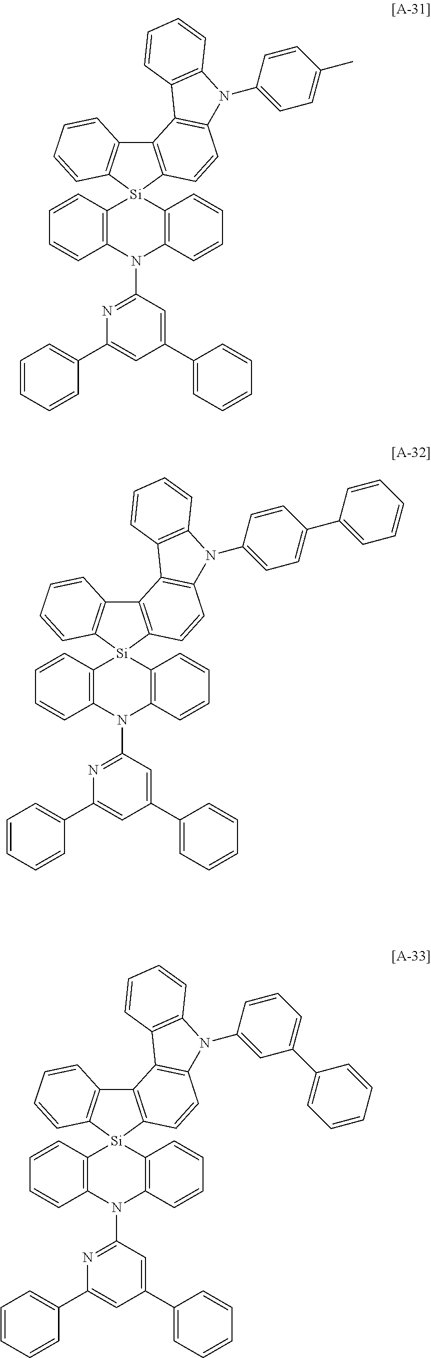 [SMILES: Cc1ccc(-n2c3ccccc3c3c4c(ccc32)[Si]2(c3ccccc3-4)c3ccccc3N(c3cc(-c4ccccc4)cc(-c4ccccc4)n3)c3ccccc32)cc1 ; c1ccc(-c2ccc(-n3c4ccccc4c4c5c(ccc43)[Si]3(c4ccccc4-5)c4ccccc4N(c4cc(-c5ccccc5)cc(-c5ccccc5)n4)c4ccccc43)cc2)cc1 ; c1ccc(-c2cccc(-n3c4ccccc4c4c5c(ccc43)[Si]3(c4ccccc4-5)c4ccccc4N(c4cc(-c5ccccc5)cc(-c5ccccc5)n4)c4ccccc43)c2)cc1]

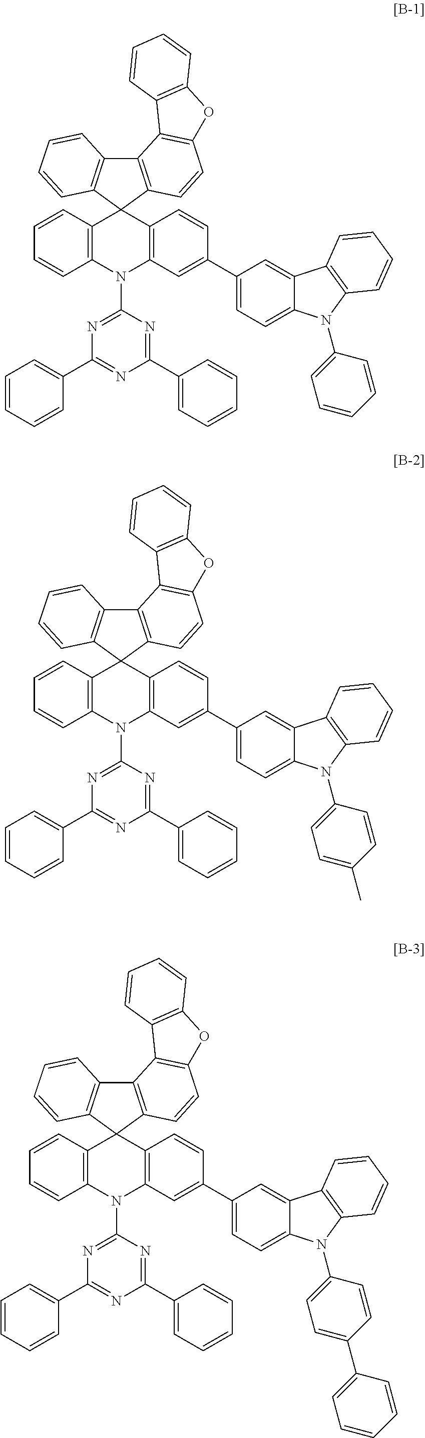 [SMILES: c1ccc(-c2nc(-c3ccccc3)nc(N3c4ccccc4C4(c5ccccc5-c5c4ccc4oc6ccccc6c54)c4ccc(-c5ccc6c(c5)c5ccccc5n6-c5ccccc5)cc43)n2)cc1 ; Cc1ccc(-n2c3ccccc3c3cc(-c4ccc5c(c4)N(c4nc(-c6ccccc6)nc(-c6ccccc6)n4)c4ccccc4C54c5ccccc5-c5c4ccc4oc6ccccc6c54)ccc32)cc1 ; c1ccc(-c2ccc(-n3c4ccccc4c4cc(-c5ccc6c(c5)N(c5nc(-c7ccccc7)nc(-c7ccccc7)n5)c5ccccc5C65c6ccccc6-c6c5ccc5oc7ccccc7c65)ccc43)cc2)cc1]

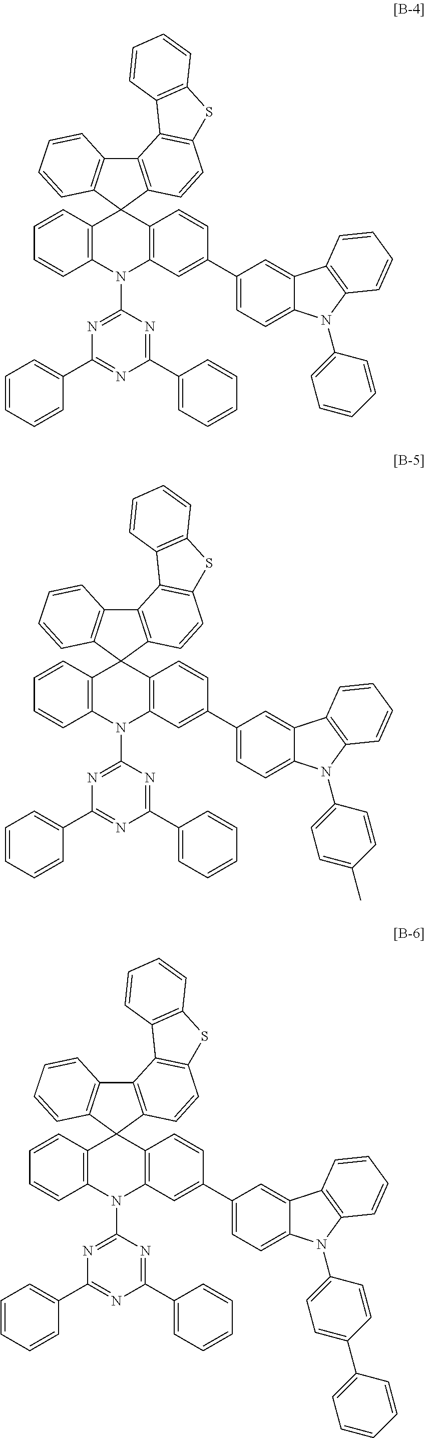 [SMILES: c1ccc(-c2nc(-c3ccccc3)nc(N3c4ccccc4C4(c5ccccc5-c5c4ccc4sc6ccccc6c54)c4ccc(-c5ccc6c(c5)c5ccccc5n6-c5ccccc5)cc43)n2)cc1 ; Cc1ccc(-n2c3ccccc3c3cc(-c4ccc5c(c4)N(c4nc(-c6ccccc6)nc(-c6ccccc6)n4)c4ccccc4C54c5ccccc5-c5c4ccc4sc6ccccc6c54)ccc32)cc1 ; c1ccc(-c2ccc(-n3c4ccccc4c4cc(-c5ccc6c(c5)N(c5nc(-c7ccccc7)nc(-c7ccccc7)n5)c5ccccc5C65c6ccccc6-c6c5ccc5sc7ccccc7c65)ccc43)cc2)cc1]

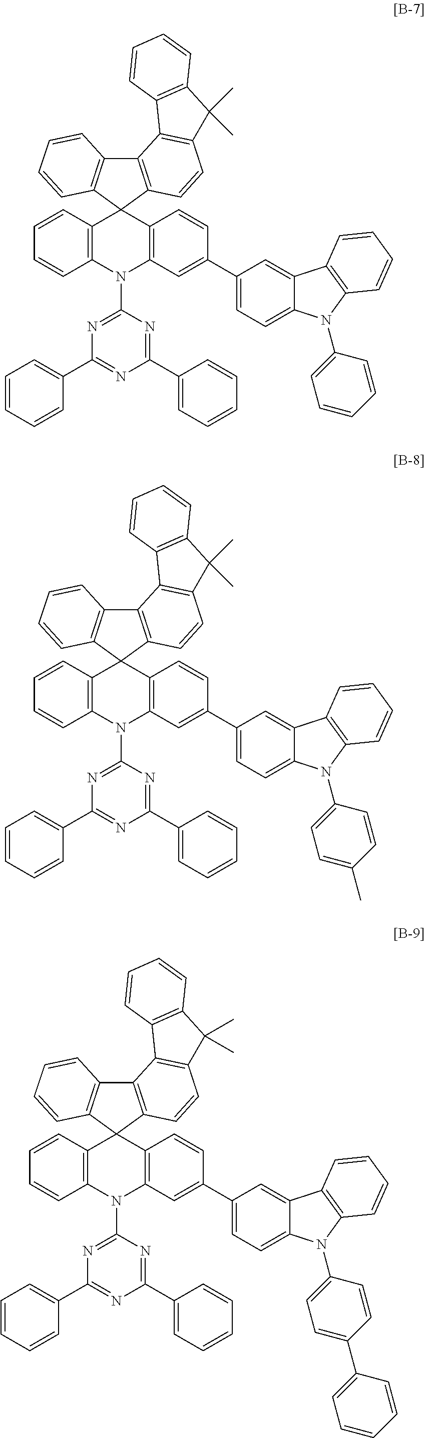 [SMILES: CC1(C)c2ccccc2-c2c1ccc1c2-c2ccccc2C12c1ccccc1N(c1nc(-c3ccccc3)nc(-c3ccccc3)n1)c1cc(-c3ccc4c(c3)c3ccccc3n4-c3ccccc3)ccc12 ; Cc1ccc(-n2c3ccccc3c3cc(-c4ccc5c(c4)N(c4nc(-c6ccccc6)nc(-c6ccccc6)n4)c4ccccc4C54c5ccccc5-c5c4ccc4c5-c5ccccc5C4(C)C)ccc32)cc1 ; CC1(C)c2ccccc2-c2c1ccc1c2-c2ccccc2C12c1ccccc1N(c1nc(-c3ccccc3)nc(-c3ccccc3)n1)c1cc(-c3ccc4c(c3)c3ccccc3n4-c3ccc(-c4ccccc4)cc3)ccc12]

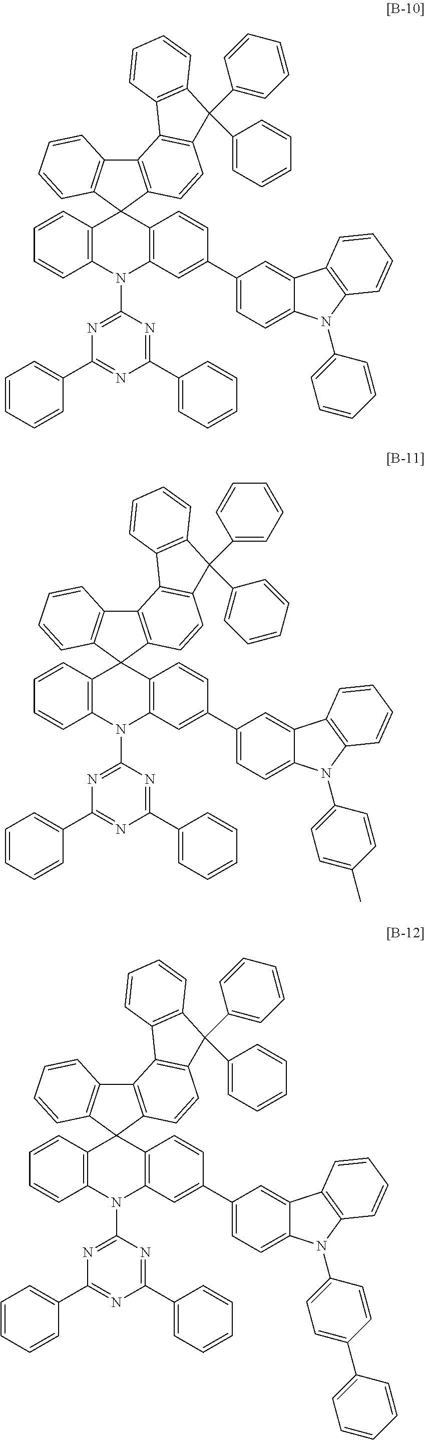 [SMILES: c1ccc(-c2nc(-c3ccccc3)nc(N3c4ccccc4C4(c5ccccc5-c5c4ccc4c5-c5ccccc5C4(c4ccccc4)c4ccccc4)c4ccc(-c5ccc6c(c5)c5ccccc5n6-c5ccccc5)cc43)n2)cc1 ; Cc1ccc(-n2c3ccccc3c3cc(-c4ccc5c(c4)N(c4nc(-c6ccccc6)nc(-c6ccccc6)n4)c4ccccc4C54c5ccccc5-c5c4ccc4c5-c5ccccc5C4(c4ccccc4)c4ccccc4)ccc32)cc1 ; c1ccc(-c2ccc(-n3c4ccccc4c4cc(-c5ccc6c(c5)N(c5nc(-c7ccccc7)nc(-c7ccccc7)n5)c5ccccc5C65c6ccccc6-c6c5ccc5c6-c6ccccc6C5(c5ccccc5)c5ccccc5)ccc43)cc2)cc1]

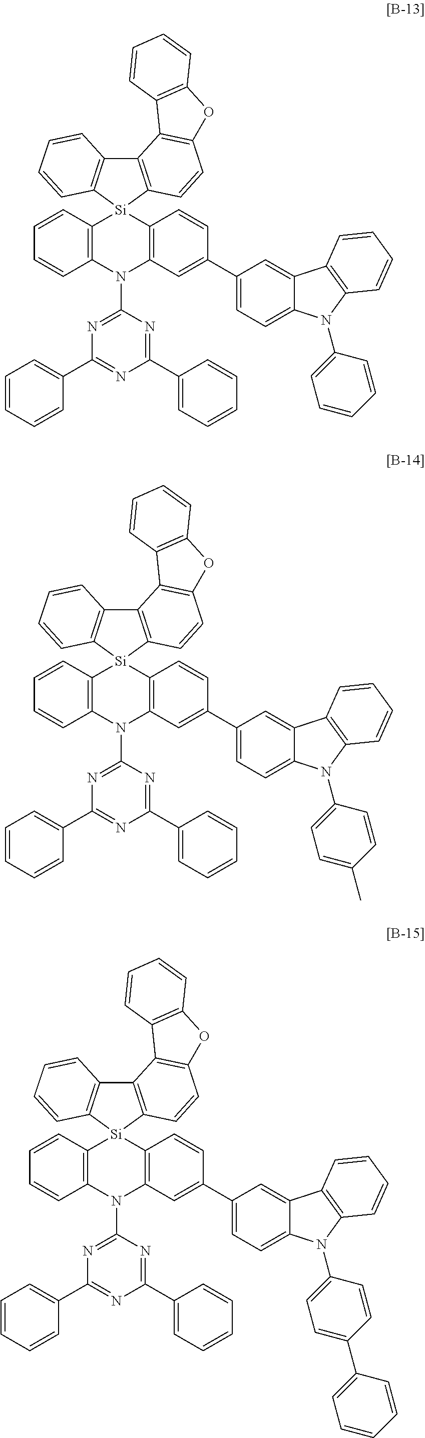 [SMILES: c1ccc(-c2nc(-c3ccccc3)nc(N3c4ccccc4[Si]4(c5ccccc5-c5c4ccc4oc6ccccc6c54)c4ccc(-c5ccc6c(c5)c5ccccc5n6-c5ccccc5)cc43)n2)cc1 ; Cc1ccc(-n2c3ccccc3c3cc(-c4ccc5c(c4)N(c4nc(-c6ccccc6)nc(-c6ccccc6)n4)c4ccccc4[Si]54c5ccccc5-c5c4ccc4oc6ccccc6c54)ccc32)cc1 ; c1ccc(-c2ccc(-n3c4ccccc4c4cc(-c5ccc6c(c5)N(c5nc(-c7ccccc7)nc(-c7ccccc7)n5)c5ccccc5[Si]65c6ccccc6-c6c5ccc5oc7ccccc7c65)ccc43)cc2)cc1]

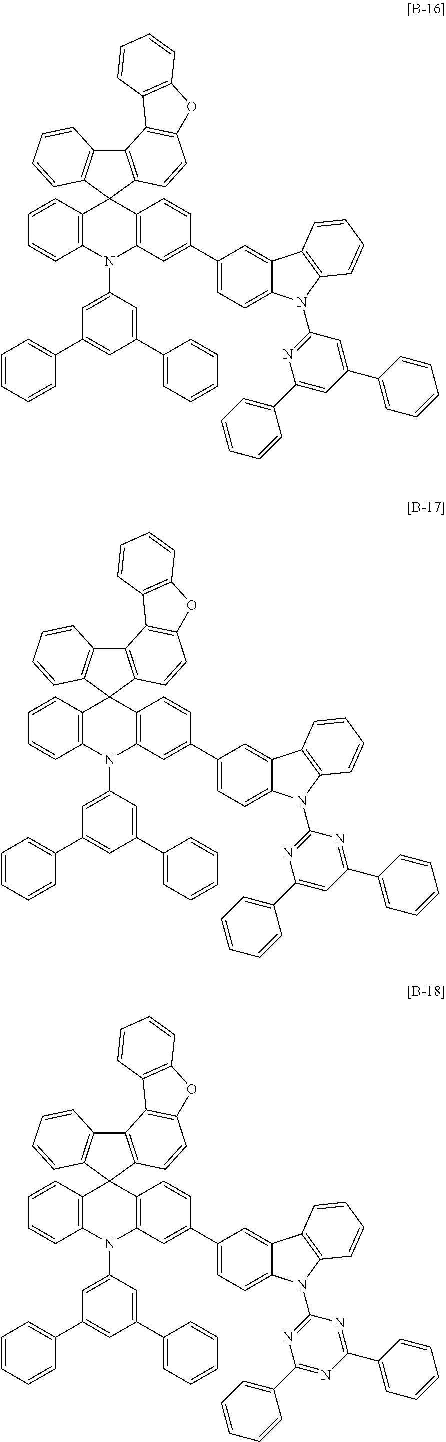 [SMILES: c1ccc(-c2cc(-c3ccccc3)cc(N3c4ccccc4C4(c5ccccc5-c5c4ccc4oc6ccccc6c54)c4ccc(-c5ccc6c(c5)c5ccccc5n6-c5cc(-c6ccccc6)cc(-c6ccccc6)n5)cc43)c2)cc1 ; c1ccc(-c2cc(-c3ccccc3)cc(N3c4ccccc4C4(c5ccccc5-c5c4ccc4oc6ccccc6c54)c4ccc(-c5ccc6c(c5)c5ccccc5n6-c5nc(-c6ccccc6)cc(-c6ccccc6)n5)cc43)c2)cc1 ; c1ccc(-c2cc(-c3ccccc3)cc(N3c4ccccc4C4(c5ccccc5-c5c4ccc4oc6ccccc6c54)c4ccc(-c5ccc6c(c5)c5ccccc5n6-c5nc(-c6ccccc6)nc(-c6ccccc6)n5)cc43)c2)cc1]

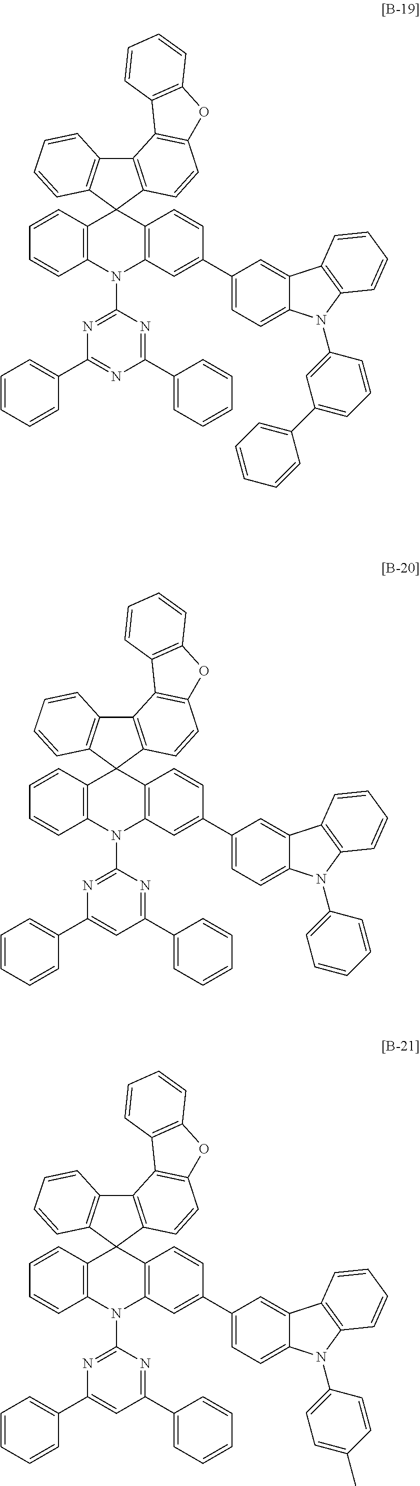 [SMILES: c1ccc(-c2cccc(-n3c4ccccc4c4cc(-c5ccc6c(c5)N(c5nc(-c7ccccc7)nc(-c7ccccc7)n5)c5ccccc5C65c6ccccc6-c6c5ccc5oc7ccccc7c65)ccc43)c2)cc1 ; c1ccc(-c2cc(-c3ccccc3)nc(N3c4ccccc4C4(c5ccccc5-c5c4ccc4oc6ccccc6c54)c4ccc(-c5ccc6c(c5)c5ccccc5n6-c5ccccc5)cc43)n2)cc1 ; Cc1ccc(-n2c3ccccc3c3cc(-c4ccc5c(c4)N(c4nc(-c6ccccc6)cc(-c6ccccc6)n4)c4ccccc4C54c5ccccc5-c5c4ccc4oc6ccccc6c54)ccc32)cc1]

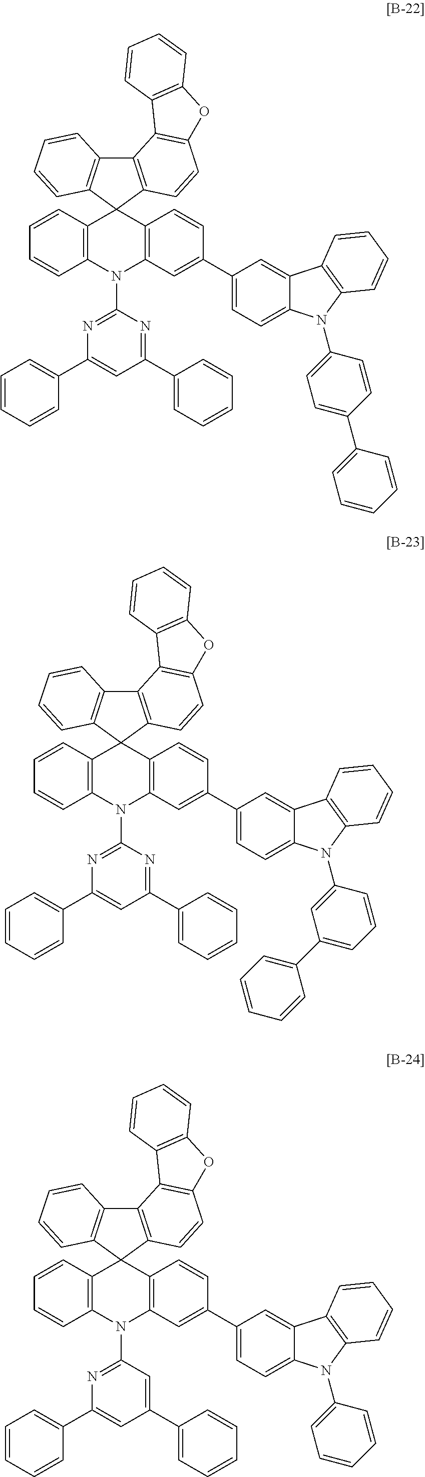 [SMILES: c1ccc(-c2ccc(-n3c4ccccc4c4cc(-c5ccc6c(c5)N(c5nc(-c7ccccc7)cc(-c7ccccc7)n5)c5ccccc5C65c6ccccc6-c6c5ccc5oc7ccccc7c65)ccc43)cc2)cc1 ; c1ccc(-c2cccc(-n3c4ccccc4c4cc(-c5ccc6c(c5)N(c5nc(-c7ccccc7)cc(-c7ccccc7)n5)c5ccccc5C65c6ccccc6-c6c5ccc5oc7ccccc7c65)ccc43)c2)cc1 ; c1ccc(-c2cc(-c3ccccc3)nc(N3c4ccccc4C4(c5ccccc5-c5c4ccc4oc6ccccc6c54)c4ccc(-c5ccc6c(c5)c5ccccc5n6-c5ccccc5)cc43)c2)cc1]

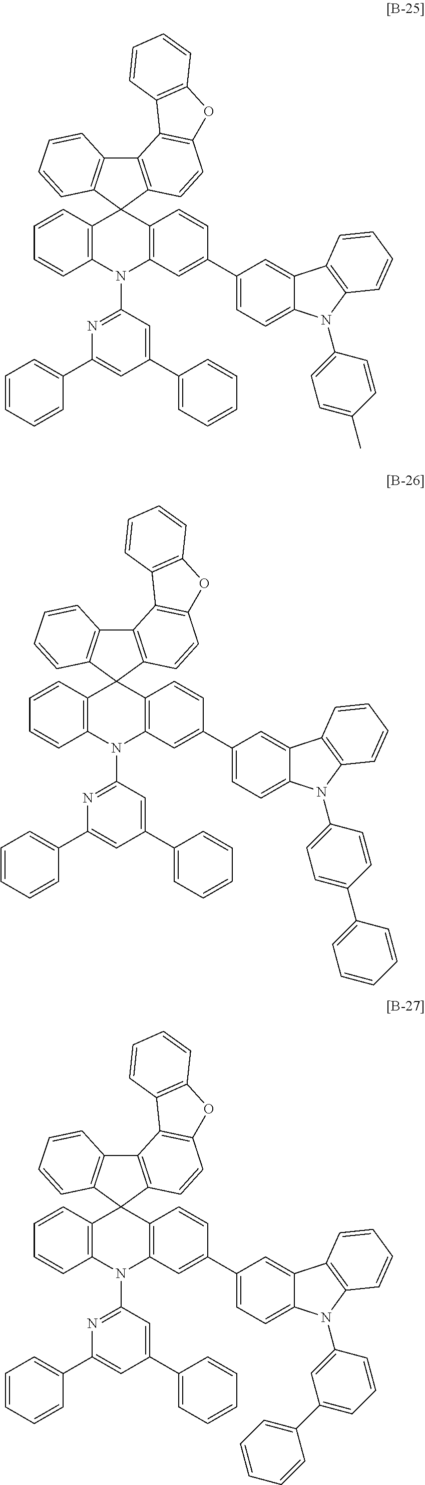 [SMILES: Cc1ccc(-n2c3ccccc3c3cc(-c4ccc5c(c4)N(c4cc(-c6ccccc6)cc(-c6ccccc6)n4)c4ccccc4C54c5ccccc5-c5c4ccc4oc6ccccc6c54)ccc32)cc1 ; c1ccc(-c2ccc(-n3c4ccccc4c4cc(-c5ccc6c(c5)N(c5cc(-c7ccccc7)cc(-c7ccccc7)n5)c5ccccc5C65c6ccccc6-c6c5ccc5oc7ccccc7c65)ccc43)cc2)cc1 ; c1ccc(-c2cccc(-n3c4ccccc4c4cc(-c5ccc6c(c5)N(c5cc(-c7ccccc7)cc(-c7ccccc7)n5)c5ccccc5C65c6ccccc6-c6c5ccc5oc7ccccc7c65)ccc43)c2)cc1]

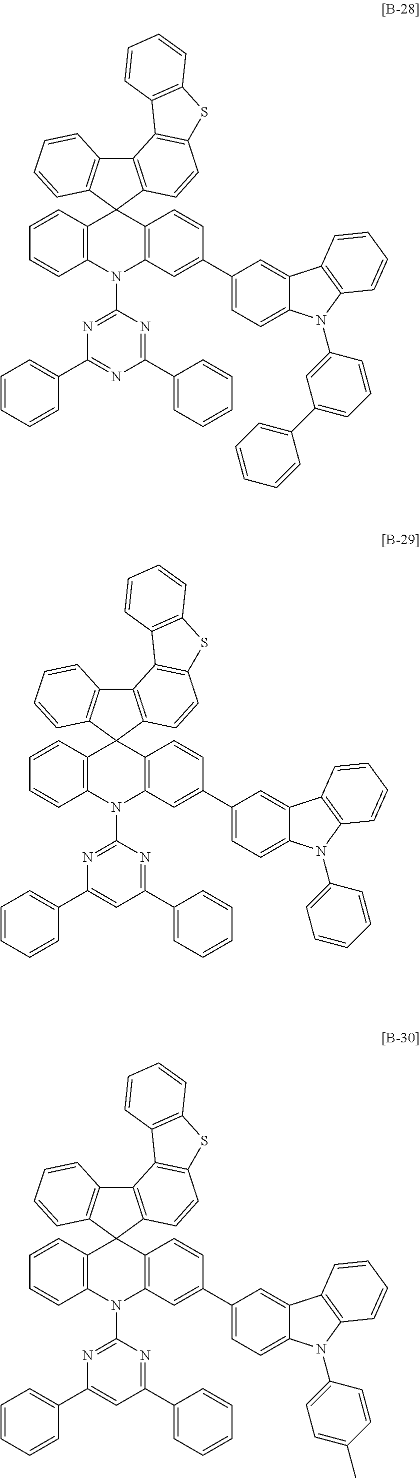 [SMILES: c1ccc(-c2cccc(-n3c4ccccc4c4cc(-c5ccc6c(c5)N(c5nc(-c7ccccc7)nc(-c7ccccc7)n5)c5ccccc5C65c6ccccc6-c6c5ccc5sc7ccccc7c65)ccc43)c2)cc1 ; c1ccc(-c2cc(-c3ccccc3)nc(N3c4ccccc4C4(c5ccccc5-c5c4ccc4sc6ccccc6c54)c4ccc(-c5ccc6c(c5)c5ccccc5n6-c5ccccc5)cc43)n2)cc1 ; Cc1ccc(-n2c3ccccc3c3cc(-c4ccc5c(c4)N(c4nc(-c6ccccc6)cc(-c6ccccc6)n4)c4ccccc4C54c5ccccc5-c5c4ccc4sc6ccccc6c54)ccc32)cc1]

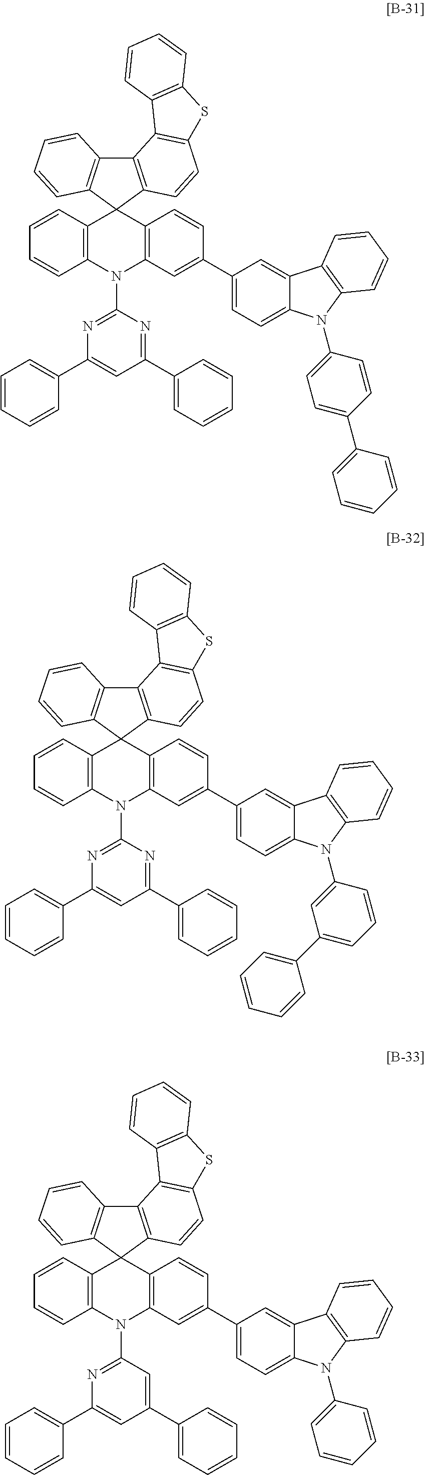 [SMILES: c1ccc(-c2ccc(-n3c4ccccc4c4cc(-c5ccc6c(c5)N(c5nc(-c7ccccc7)cc(-c7ccccc7)n5)c5ccccc5C65c6ccccc6-c6c5ccc5sc7ccccc7c65)ccc43)cc2)cc1 ; c1ccc(-c2cccc(-n3c4ccccc4c4cc(-c5ccc6c(c5)N(c5nc(-c7ccccc7)cc(-c7ccccc7)n5)c5ccccc5C65c6ccccc6-c6c5ccc5sc7ccccc7c65)ccc43)c2)cc1 ; c1ccc(-c2cc(-c3ccccc3)nc(N3c4ccccc4C4(c5ccccc5-c5c4ccc4sc6ccccc6c54)c4ccc(-c5ccc6c(c5)c5ccccc5n6-c5ccccc5)cc43)c2)cc1]

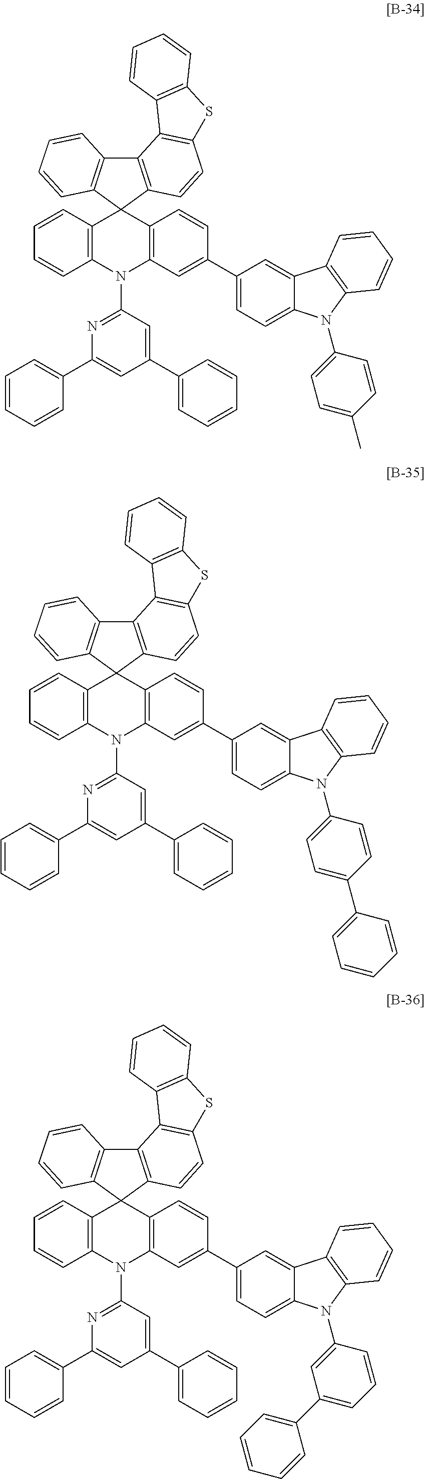 [SMILES: Cc1ccc(-n2c3ccccc3c3cc(-c4ccc5c(c4)N(c4cc(-c6ccccc6)cc(-c6ccccc6)n4)c4ccccc4C54c5ccccc5-c5c4ccc4sc6ccccc6c54)ccc32)cc1 ; c1ccc(-c2ccc(-n3c4ccccc4c4cc(-c5ccc6c(c5)N(c5cc(-c7ccccc7)cc(-c7ccccc7)n5)c5ccccc5C65c6ccccc6-c6c5ccc5sc7ccccc7c65)ccc43)cc2)cc1 ; c1ccc(-c2cccc(-n3c4ccccc4c4cc(-c5ccc6c(c5)N(c5cc(-c7ccccc7)cc(-c7ccccc7)n5)c5ccccc5C65c6ccccc6-c6c5ccc5sc7ccccc7c65)ccc43)c2)cc1]

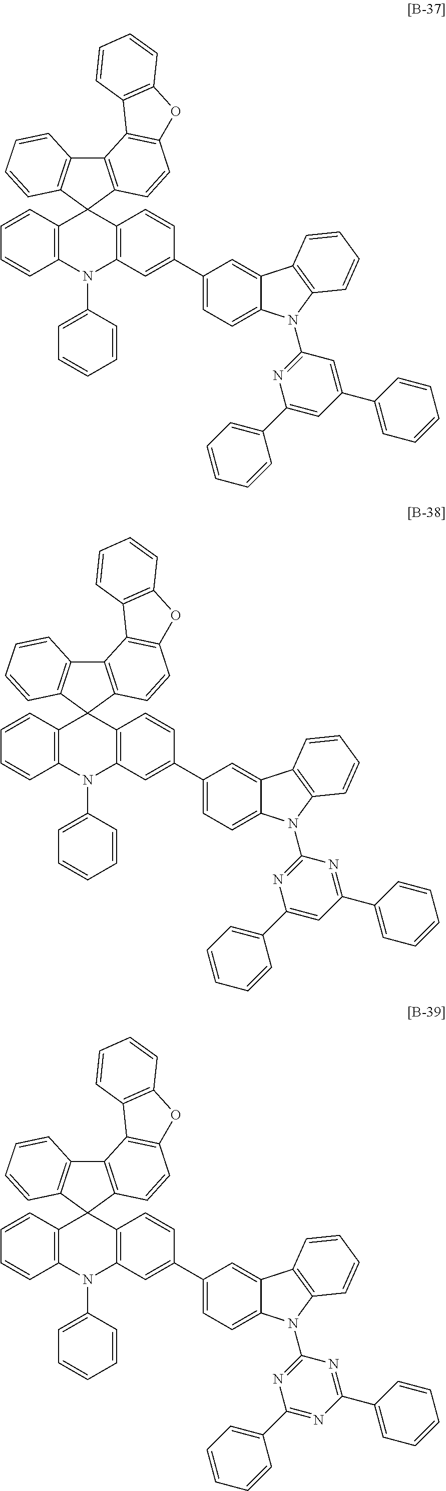 [SMILES: c1ccc(-c2cc(-c3ccccc3)nc(-n3c4ccccc4c4cc(-c5ccc6c(c5)N(c5ccccc5)c5ccccc5C65c6ccccc6-c6c5ccc5oc7ccccc7c65)ccc43)c2)cc1 ; c1ccc(-c2cc(-c3ccccc3)nc(-n3c4ccccc4c4cc(-c5ccc6c(c5)N(c5ccccc5)c5ccccc5C65c6ccccc6-c6c5ccc5oc7ccccc7c65)ccc43)n2)cc1 ; c1ccc(-c2nc(-c3ccccc3)nc(-n3c4ccccc4c4cc(-c5ccc6c(c5)N(c5ccccc5)c5ccccc5C65c6ccccc6-c6c5ccc5oc7ccccc7c65)ccc43)n2)cc1]

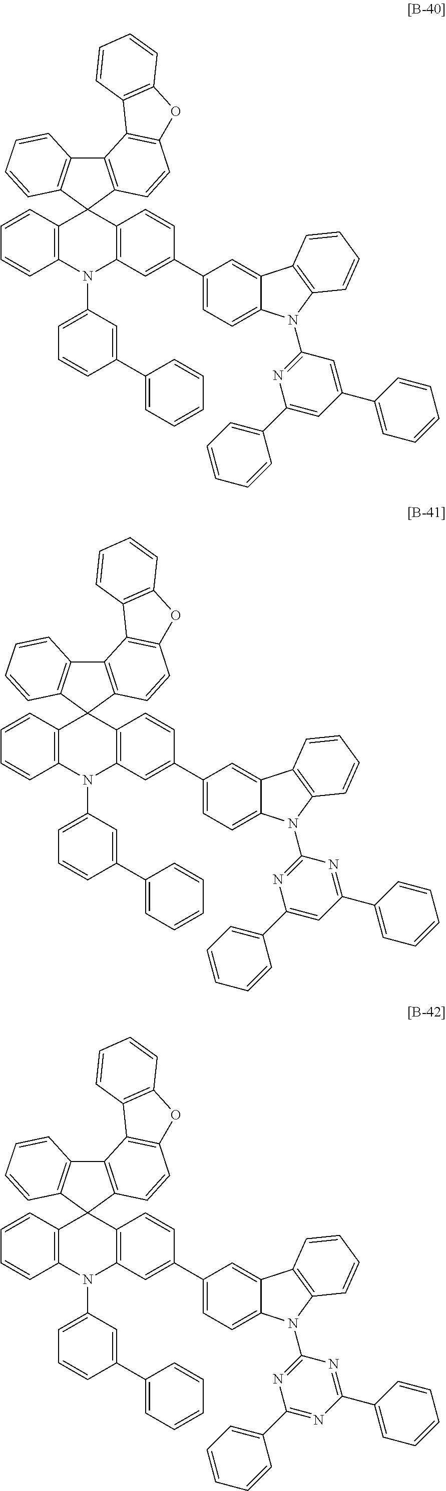 [SMILES: c1ccc(-c2cccc(N3c4ccccc4C4(c5ccccc5-c5c4ccc4oc6ccccc6c54)c4ccc(-c5ccc6c(c5)c5ccccc5n6-c5cc(-c6ccccc6)cc(-c6ccccc6)n5)cc43)c2)cc1 ; c1ccc(-c2cccc(N3c4ccccc4C4(c5ccccc5-c5c4ccc4oc6ccccc6c54)c4ccc(-c5ccc6c(c5)c5ccccc5n6-c5nc(-c6ccccc6)cc(-c6ccccc6)n5)cc43)c2)cc1 ; c1ccc(-c2cccc(N3c4ccccc4C4(c5ccccc5-c5c4ccc4oc6ccccc6c54)c4ccc(-c5ccc6c(c5)c5ccccc5n6-c5nc(-c6ccccc6)nc(-c6ccccc6)n5)cc43)c2)cc1]

The compound for an organic optoelectronic device has a maximum light emitting wavelength in a range of about 320 to about 520 nm and a triplet excited energy (T1) ranging from greater than or equal to about 2.0 eV, and specifically, from about 2.0 to about 4.0 eV, and thus may well transport a host charge having high triplet excited energy to a dopant and increase luminous efficiency of the dopant, and is also freely adjusted regarding HOMO and LUMO energy levels and decreases a driving voltage, and accordingly may be usefully applied as a host material or a charge transport material.

In addition, the compound for an organic optoelectronic device has photoactive and electrical activities, and thus may be usefully applied for a nonlinear optic material, an electrode material, a discolored material, a light switch, a sensor, a module, a wave guide, an organic transistor, a laser, a light absorbent, a dielectric material, a separating membrane, and the like.

The compound for an organic optoelectronic device including the compounds has a glass transition temperature of greater than or equal to 90° C. and a thermal decomposition temperature of greater than or equal to 400° C., indicating improved thermal stability. Thereby, it is possible to produce an organic optoelectronic device having high efficiency.

The compound for an organic optoelectronic device including the compounds may play a role of emitting light or injecting and/or transporting electrons, and may also act as a light emitting host with an appropriate dopant. In other words, the compound for an organic optoelectronic device may be used as a phosphorescent or fluorescent host material, a blue light emitting dopant material, or an electron transport material.

Since the compound for an organic optoelectronic device according to one embodiment is used for an organic thin layer, and it may improve the life-span characteristic, efficiency characteristic, electrochemical stability, and thermal stability of an organic optoelectronic device, and decrease the driving voltage.

Further, according to another embodiment, an organic optoelectronic device that includes the compound for an organic optoelectronic device is provided. The organic optoelectronic device may include an organic photoelectric device, an organic light emitting diode, an organic solar cell, an organic transistor, an organic photoconductor drum, an organic memory device, and the like. Particularly, the compound for an organic optoelectronic device according to one embodiment may be included in an electrode or an electrode buffer layer in an organic solar cell to improve the quantum efficiency, and it may be used as an electrode material for a gate, a source-drain electrode, or the like in the organic transistor.

Hereinafter, an organic light emitting diode is described.

According to another embodiment of the present invention, an organic light emitting diode includes an anode, a cathode, and at least one organic thin layer between the anode and the cathode, and at least one organic thin layer may include the compound for an organic optoelectronic device according to one embodiment of the present invention.

The organic thin layer that may include the compound for an organic optoelectronic device may include a layer selected from the group consisting of an emission layer, a hole transport layer (HTL), a hole injection layer (HIL), an electron transport layer (ETL), an electron injection layer (EIL), a hole blocking layer, and a combination thereof. The at least one layer includes the compound for an organic optoelectronic device according to one embodiment. Particularly, the compound for an organic optoelectronic device according to one embodiment may be included in a hole transport layer (HTL) or a hole injection layer (HIL). In addition, when the compound for an organic optoelectronic device is included in the emission layer, the compound for an organic optoelectronic device may be included as a phosphorescent or fluorescent host, and particularly, as a fluorescent blue dopant material.

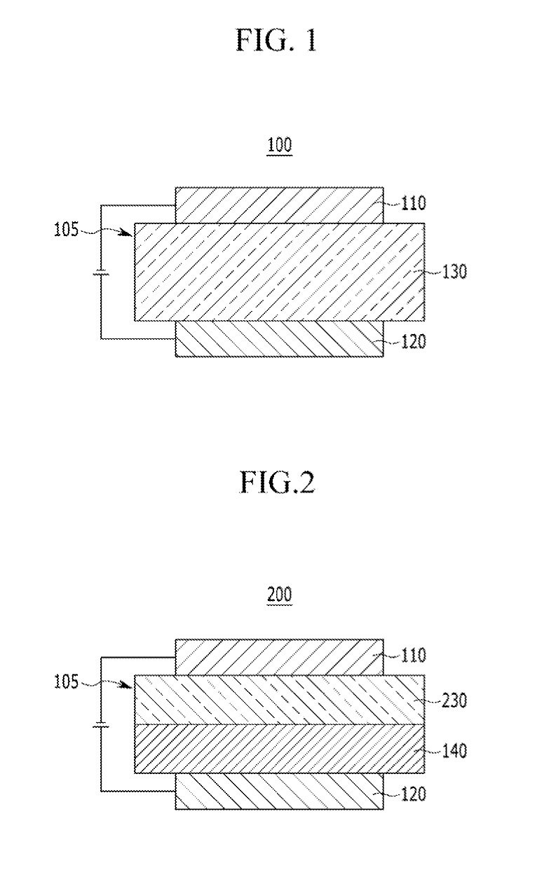

FIGS. 1 to 5 are cross-sectional views showing organic light emitting diodes including the compound for an organic optoelectronic device according to one embodiment of the present invention.

Referring to FIGS. 1 to 5, organic light emitting diodes 100, 200, 300, 400, and 500 according to one embodiment include at least one organic thin layer 105 interposed between an anode 120 and a cathode 110.

The anode 120 includes an anode material laving a large work function to help hole injection into an organic thin layer. The anode material includes: a metal such as nickel, platinum, vanadium, chromium, copper, zinc, and gold, or alloys thereof; a metal oxide such as zinc oxide, indium oxide, indium tin oxide (ITO), and indium zinc oxide (IZO); a combination of a metal and an oxide such as ZnO:Al and SnO₂:Sb; or a conductive polymer such as poly(3-methylthiophene), poly[3,4-(ethylene-1,2-dioxy)thiophene] (PEDT), polypyrrole, and polyaniline, but is not limited thereto. It is preferable to include a transparent electrode including indium tin oxide (ITO) as an anode.

The cathode 110 includes a cathode material having a small work function to help electron injection into an organic thin layer. The cathode material includes: a metal such as magnesium, calcium, sodium, potassium, titanium, indium, yttrium, lithium, gadolinium, aluminum, silver, tin, and lead, or alloys thereof; or a multi-layered material such as LiF/Al, Liq/Al, LiO₂/Al, LiF/Ca, LiF/Al, and BaF₂/Ca, but is not limited thereto. It is preferable to include a metal electrode including aluminum as a cathode.

First, referring to FIG. 1, the organic light emitting diode 100 includes an organic thin layer 105 including only an emission layer 130.

Referring to FIG. 2, a double-layered organic light emitting diode 200 includes an organic thin layer 105 including an emission layer 230 including an electron transport layer (ETL), and a hole transport layer (HTL) 140. As shown in FIG. 2, the organic thin layer 105 includes a double layer of the emission layer 230 and the hole transport layer (HTL) 140. The emission layer 230 also functions as an electron transport layer (ETL), and the hole transport layer (HTL) 140 layer has an improved binding property with a transparent electrode such as ITO or an improved hole transport capability.

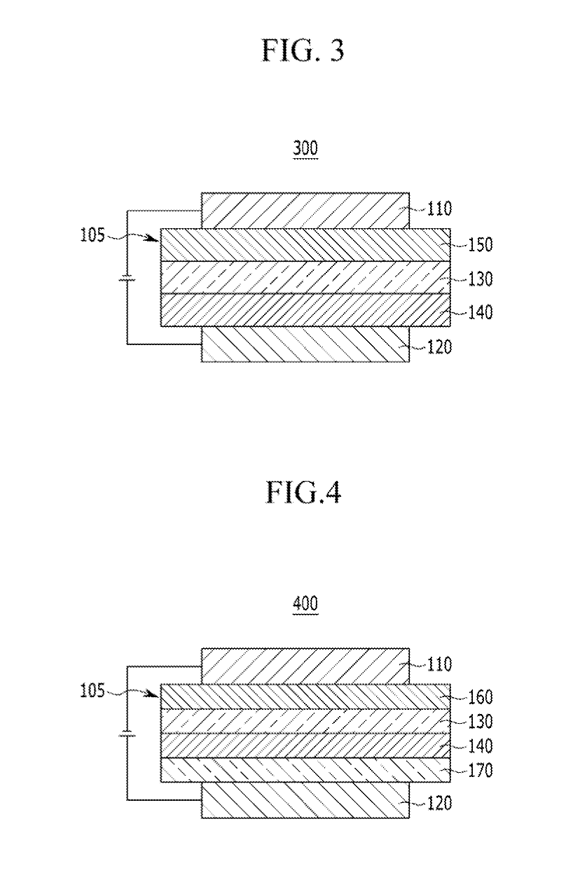

Referring to FIG. 3, a three-layered organic light emitting diode 300 includes an organic thin layer 105 including an electron transport layer (ETL) 150, an emission layer 130, and a hole transport layer (HTL) 140. The emission layer 130 is independently installed, and layers having an improved electron transport capability or an improved hole transport capability are separately stacked.

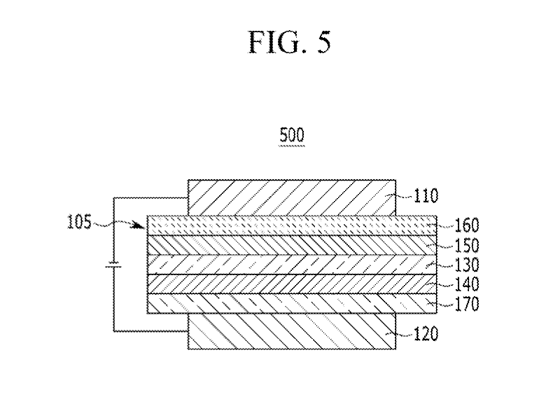

Referring to FIG. 4, a four-layered organic light emitting diode 400 includes an organic thin layer 105 including an electron injection layer (EIL) 160, an emission layer 130, a hole transport layer (HTL) 140, and a hole injection layer (HIL) 170 for adherence with the cathode made of ITO.

Referring to FIG. 5, a five-layered organic light emitting diode 500 includes an organic thin layer 105 including an electron transport layer (ETL) 150, an emission layer 130, a hole transport layer (HTL) 140, and a hole injection layer (HIL) 170, and further includes an electron injection layer (EIL) 160 to achieve a low voltage.

In FIGS. 1 to 5, the organic thin layer 105 including at least one selected from the group consisting of an electron transport layer (ETL) 150, an electron injection layer (EIL) 160, emission layers 130 and 230, a hole transport layer (HTL) 140, a hole injection layer (HIL) 170, and combinations thereof includes a compound for an organic optoelectronic device. The compound for an organic optoelectronic device may be used for an electron transport layer (ETL) 150 including the electron transport layer (ETL) 150 or electron injection layer (EIL) 160. When it is used for the electron transport layer (ETL), it is possible to provide an organic light emitting diode having a more simple structure because it does not require an additional hole blocking layer (not shown).

Furthermore, when the compound for an organic optoelectronic device is included in the emission layers 130 and 230, the compound for the organic optoelectronic device may be included as a phosphorescent or fluorescent host or a fluorescent blue dopant.

The organic light emitting diode may be fabricated by: forming an anode on a substrate; forming an organic thin layer in accordance with a dry coating method such as evaporation, sputtering, plasma plating, and ion plating, or a wet coating method such as spin coating, dipping, and flow coating; and providing a cathode thereon.

Another embodiment of the present invention provides a display device including the organic light emitting diode according to the embodiment.

Hereinafter, the embodiments are illustrated in more detail with reference to examples. These examples, however, are not in any sense to be interpreted as limiting the scope of the invention.

(Preparation of Compound for Organic Optoelectronic Device)

### Example 1

### Synthesis of Compound Represented by Chemical Formula (A-1)

A compound represented by the above Chemical Formula (A-1) as specific examples of a compound for an organic optoelectronic device according to the present invention was synthesized according to the following Reaction Scheme 1.

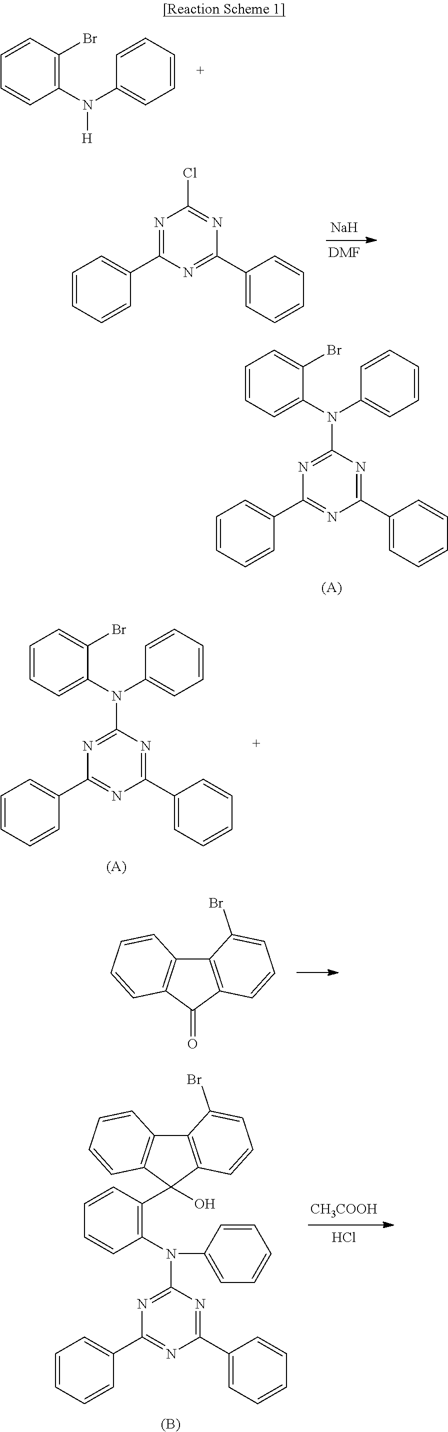

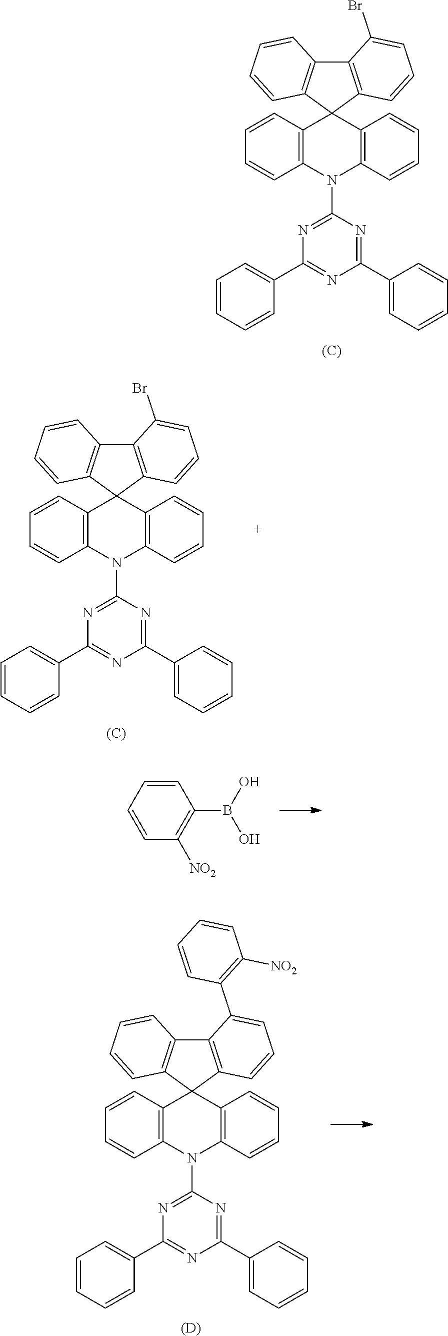

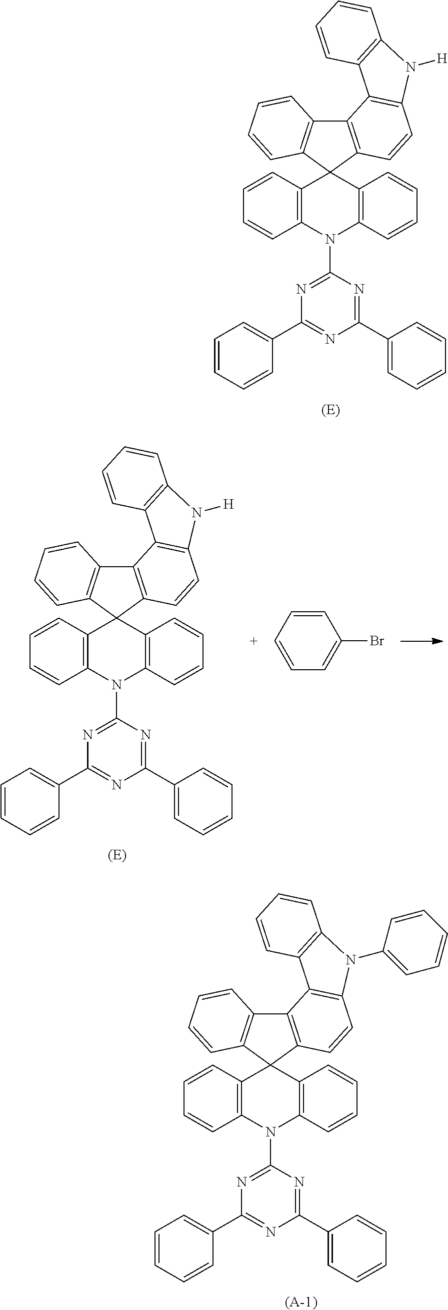

First Step; Synthesis of Compound (A)

30 g (120 mmol) of 2-bromo aniline, 38.8 g (150 mmol) of 1-chloro-3,5-phenyl triazine, and 7.3 g (300 mmol) of sodium hydride were put in 900 ml of dimethyl formaldehyde in a 2000 mL round flask, and the mixture was agitated at room temperature for 24 hours. After removing non-reaction sodium hydride by slowly dropping the reaction solution to distilled water, the reaction solution was poured into an excessive water of distilled water, and the mixture was filtered. The obtained solid was dissolved in an excessive amount of methylene chloride, the solution was hot-filtered, methylene chloride was removed therefrom again, and a solid obtained by precipitating the resultant in methanol was filtered, obtaining 40.5 g of a compound (A) (a yield of 70%).

The elemental analysis result of the compound (A) was provided as follows.

calcd. C₂₇H₁₉BrN₄: C, 67.65; H, 4.00; Br, 16.67; N, 11.69. found: C, 67.35; H, 4.03; N, 10.88.

Second step: Synthesis of Compound (C)

30 g (62.5 mmol) of the compound (A) was put in a 500 mL round flask and dissolved in 300 mL of tetrahydrofuran after making its reaction atmosphere be in a nitrogen state, and a temperature around a reactor was maintained at −78° C. Then, 25 mL of 2.5M-normal butyl lithium was slowly added thereto in a dropwise fashion. When the addition was complete, the mixture was agitated for 30 minutes, and a solution obtained by dissolving 16.2 g (62.5 mmol) of 2-bromo fluoreneol in 200 mL of purified tetrahydrofuran was slowly added thereto. The reaction solution was agitated for about one hour while maintained at −78° C. and then, heated up to room temperature and kept being agitated until the next day morning. After completing a reaction by adding a 5 wt % sodium bicarbonate aqueous solution to the reaction solution, the resultant was separately extracted by using methylene chloride. The obtained organic layer was distilled under a reduced pressure after removing water remaining there with magnesium sulfate, obtaining an intermediate (B). The unpurified intermediate (B) was put in 400 mL of acetic acid, hydrochloric acid in a catalyst amount was added thereto, and the mixture was agitated for 12 hours at a reflux temperature. When the reaction was complete, 24 g of a compound (C) was obtained through column chromatography (a yield of 62%).

The elemental analysis result of the compound (C) was provided as follows.

calcd. C₄₀H₂₅BrN₄: C, 74.88; H, 3.93; Br, 12.45; N, 8.73. found: C, 74.65; H, 3.91; N, 8.75

Third step: Synthesis of Compound (E)

20 g (31.1 mmol) of the compound (C), 6.3 g (31.1 mmol) of a 2-nitrobenzene boronic acid compound and 1.8 g (1.2 mmol) of tetrakis triphenyl phosphine were suspended in 400 ml of toluene/tetrahydrofuran, 400 ml of a 2 M potassium carbonate aqueous solution was added thereto, and the mixture was heated and refluxed under a nitrogen stream for 24 hours. The reaction solution was added to 2000 ml of MeOH, a crystallized solid therein was filtered and dissolved in monochlorobenzene, and the solution was filtered with silica gel/Celite. After removing an organic solvent in an appropriate amount therefrom, the residue was recrystallized in MeOH, obtaining 15 g of a compound (A-1) (a yield of 70%).

15 g (20 mmol) of the compound (D) was agitated with 18 g (100 mmol) of triethyl phosphite under a nitrogen stream for one night. After removing untreated triethyl phosphite by a vacuum distillation method, 8.52 g (13.1 mmol) of a compound (E) was obtained through column chromatography for a solid remaining in the flask.

The elemental analysis result of the compound (E) was provided as follows.

calcd. C₄₆H₂₉N₅: C, 84.77; H, 4.48; N, 10.75. found: C, 84.73; H, 4.45; N, 10.78

Fourth step: Synthesis of Compound (A-1)

8.5 g (13 mmol) of the compound represented by compound (E), 2.5 g (15.6 mmol) of bromo benzene and 2.8 g (19.5 mmol) of potassium carbonate were suspended in 250 ml of DMSO, 0.5 g (0.3 mmol) of 1,10-phenanthroline and 0.3 g (0.3 mmol) of copper chloride were added, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 5.7 g of a compound (A-1) (a yield of 60%).

The elemental analysis result of the compound (A-1) was provided as follows.

calcd. C₅₂H₃₃N₅: C, 85.81; H, 4.57; N, 9.62. found: C, 85.76; H, 4.60; N, 9.58

### Example 2

### Synthesis of Compound Represented by Chemical Formula (A-4)

A compound represented by the above Chemical Formula (A-4) as specific examples of a compound for an organic optoelectronic device of the present invention was synthesized according to the following Reaction Scheme 2.

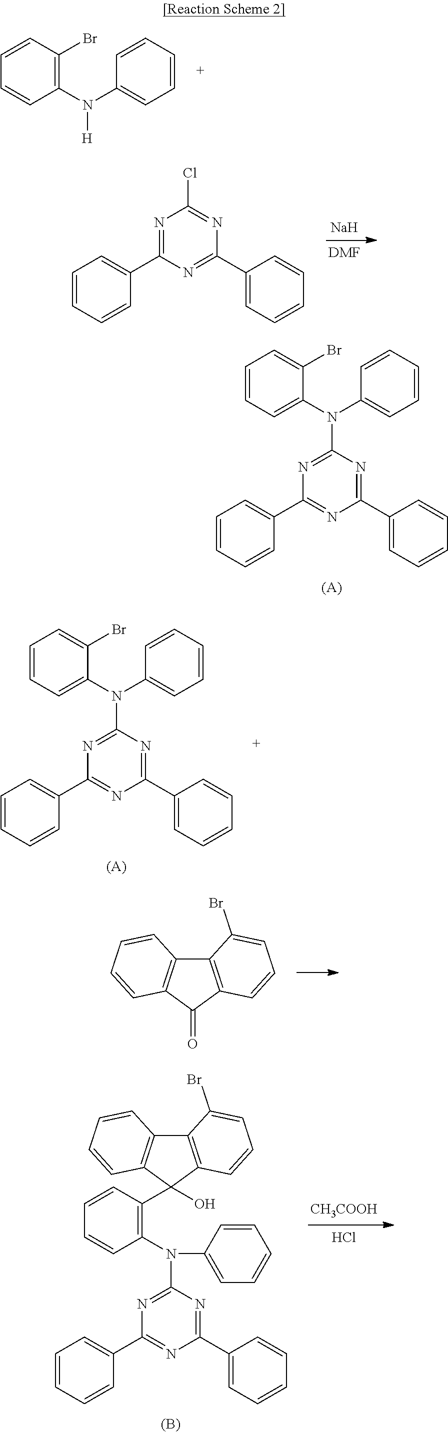

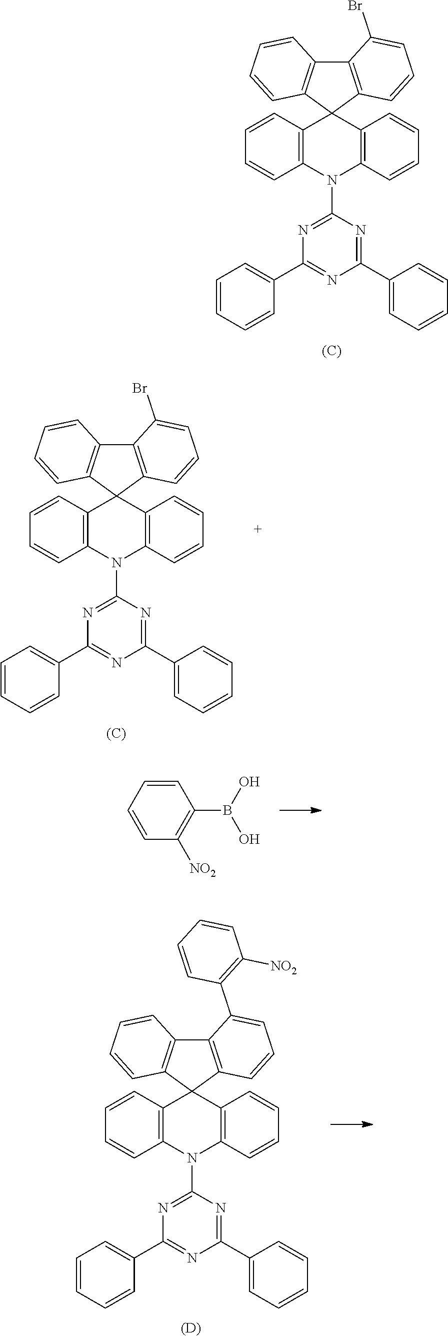

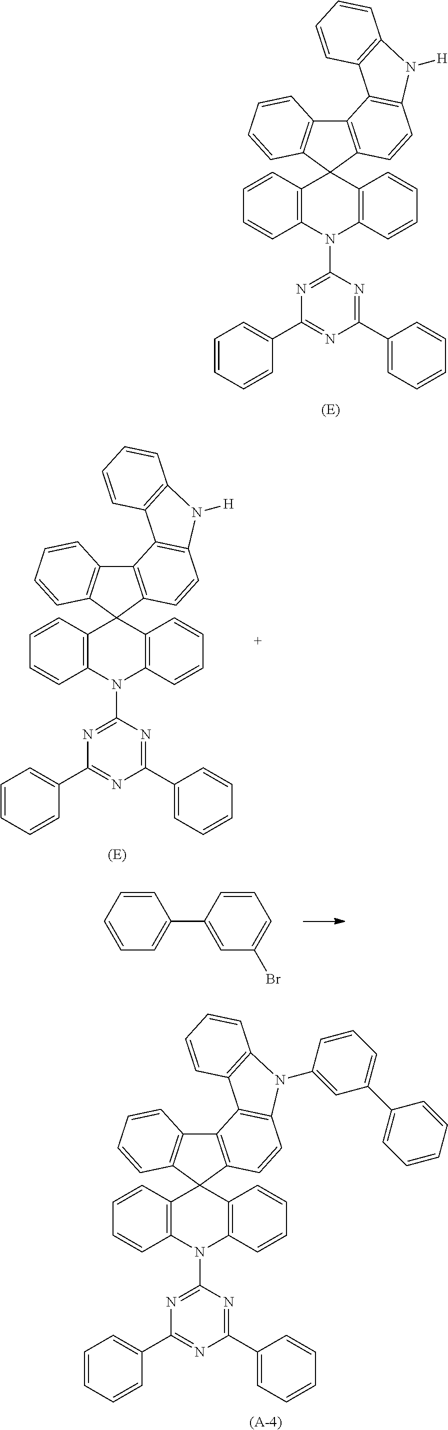

First step: Synthesis of Compound (A-4)

8.5 g (13 mmol) of the compound represented by the compound (E) synthesized through the first step, the second step and the third step in Example 1, 3 g (15.6 mmol) of 3-bromo biphenyl and 2.8 g (19.5 mmol) of potassium carbonate were suspended in 250 ml of DMSO, 0.5 g (0.3 mmol) of 1,10-phenanthroline and 0.3 g (0.3 mmol) of copper chloride were added, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 6.3 g of a compound (A-4) (a yield of 60%).

The elemental analysis result of the compound (A-4) was provided as follows.

calcd. C₅₈H₃₇N₅: C, 86.65; H, 4.64; N, 8.71. found: C, 86.67; H, 4.61; N, 8.73

### Example 3

### Synthesis of Compound Represented by Chemical Formula (A-5)

A compound represented by the above Chemical Formula (A-5) as specific examples of a compound for an organic optoelectronic device of the present invention was synthesized according to the following Reaction Scheme 3.

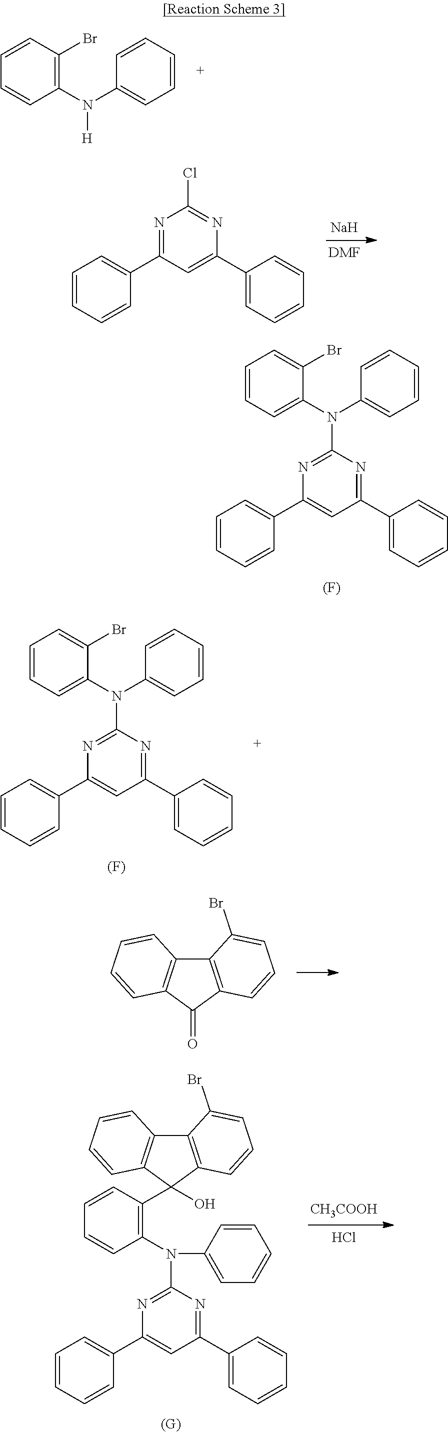

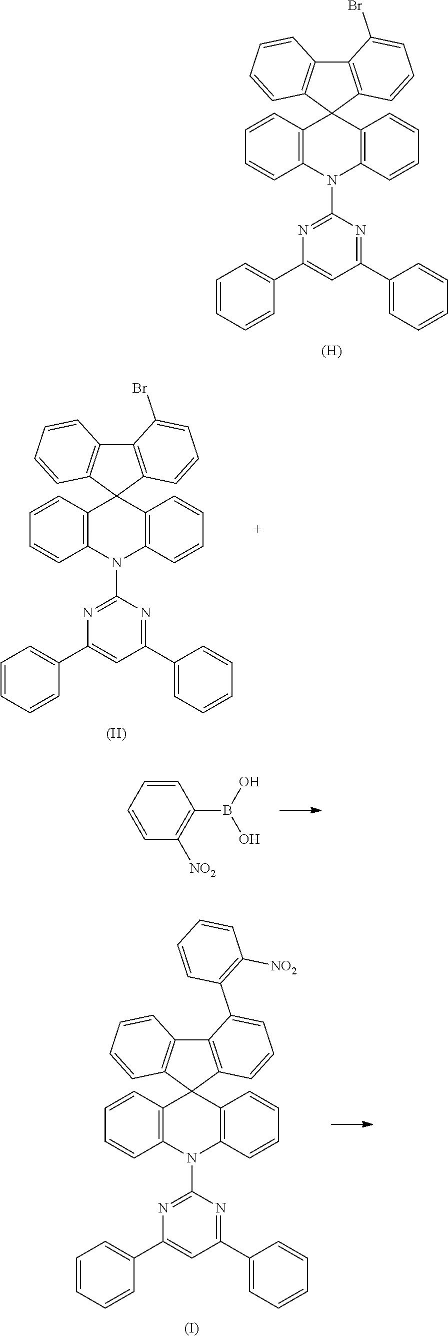

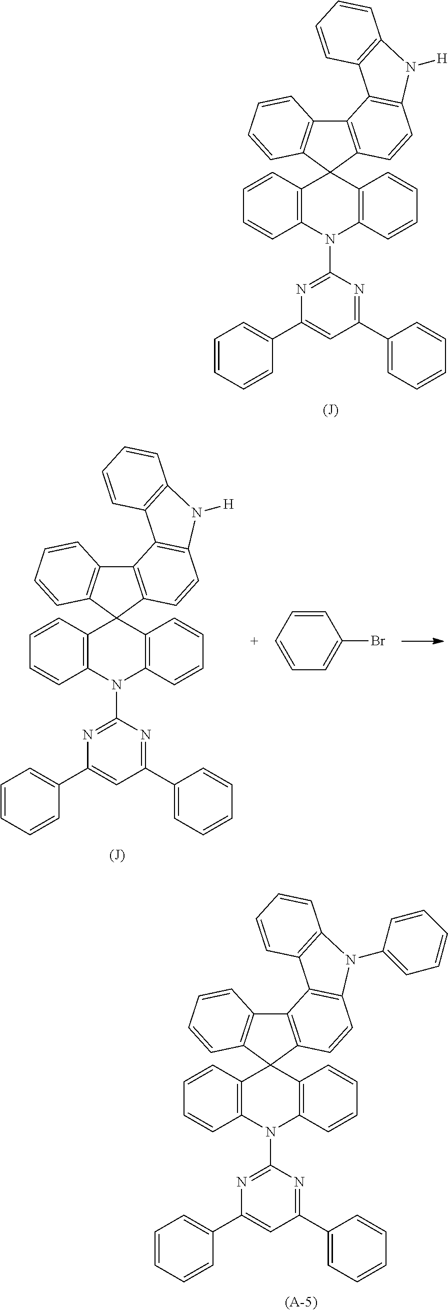

First Step; Synthesis of Compound (F)

30 g (120 mmol) of 2-bromo aniline, 38.8 g (150 mmol) of 1-chloro-3,5-phenyl pyrimidine, and 7.3 g (300 mmol) of sodium hydride were agitated in 900 ml of dimethyl formaldehyde in a 2000 mL round flask at room temperature for 24 hours. After removing non-reaction sodium hydride by slowly dropping the reaction solution to distilled water, the reaction solution was poured into an excessive amount of distilled water again, and then, the mixture was filtered. The obtained solid was dissolved in an excessive amount of methylene chloride, the solution was hot-filtered, methylene chloride was removed therefrom again, and a solid obtained by precipitating the residue in methanol was filtered, obtaining 36.5 g of a compound (FD) (a yield of 66%).

The elemental analysis result of the compound (FD) was provided as follows.

calcd. C₂₈H₂₀BrN₃: C, 70.30; H, 4.21; Br, 16.70; N, 8.78. found: C, 70.32; H, 4.20; N, 8.90

Second step: Synthesis of Compound (H)

30 g (62.5 mmol) of the compound (F) was put in a 500 mL round flask and then, dissolved in 300 mL of purified tetrahydrofuran after making its reaction atmosphere be in a nitrogen state, and a temperature around a reactor was maintained at −78° C. Then, 25 mL of 2.5 M-normal butyl lithium was slowly added thereto in a dropwise fashion. When the addition was complete, the mixture was agitated for 30 minutes, and a solution obtained by dissolving 16.2 g (62.5 mmol) of 4-bromo fluoreneol in 200 mL of purified tetrahydrofuran was slowly added thereto. The reaction solution was agitated for about one hour while maintained at −78° C. and then, heated up to room temperature and kept being agitated until the next day morning. After completing the reaction by adding a 5 wt % sodium bicarbonate aqueous solution to the reaction solution, the mixture was separately extracted by using methylene chloride. The obtained organic layer was distilled under a reduced pressure after removing water remaining there by using magnesium sulfate, obtaining an intermediate (G). The unpurified intermediate (G) was put in 400 mL of acetic acid, hydrochloric acid in a catalyst amount was added thereto, and the mixture was agitated at a reflux temperature for 12 hours. When the reaction was complete, 28 g of a compound (H) was obtained through column chromatography (a yield of 64%).

The elemental analysis result of the compound (H) was provided as follows.

calcd. C₄₁H₂₆BrN₃: C, 76.88; H, 4.09; Br, 12.47; N, 6.56. found: C, 76.72; H, 4.15; N, 6.61

Third step: Synthesis of Compound (J)

20 g (31.1 mmol) of the compound represented by the compound (H), 6.3 g (31.1 mmol) of a 2-nitrobenzene boronic acid compound and 1.8 g (1.2 mmol) of tetrakis tetrakis triphenyl phosphine were suspended in 400 ml of toluene/tetrahydrofuran, 400 ml of a 2M potassium carbonate aqueous solution was added, and the mixture was heated and refluxed under a nitrogen stream for 24 hours. The reaction solution was added to 2000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 14.7 g of a compound (I) (a yield of 70%).

14.7 g (20 mmol) of the compound (I) was agitated with 18 g (100 mmol) of triethyl phosphite under a nitrogen stream for one night. After removing untreated triethyl phosphite by a vacuum distillation method, 8.5 g (13.2 mmol) of a compound (J) was obtained through column chromatography for a solid remaining in the flask.

The elemental analysis result of the compound (J) was provided as follows.

calcd. C₄₆H₂₉N₅: C, 84.77; H, 4.48; N, 10.75. found: C, 84.73; H, 4.45; N, 10.78

Fourth step: Synthesis of Compound (A-5)

8.5 g (13 mmol) of the compound represented by compound (J), 2.5 g (15.6 mmol) of bromo benzene and 2.8 g (19.5 mmol) of potassium carbonate were suspended in 250 ml of DMSO, 0.5 g (0.3 mmol) of 1,10-phenanthroline and 0.3 g (0.3 mmol) of copper chloride, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 5.5 g of a compound (A-5) (a yield of 60%).

The elemental analysis result of the compound (A-5) was provided as follows.

calcd. C₅₃H₃₄N₄: C, 87.58; H, 4.71; N, 7.71. found: C, 87.54; H, 4.68; N, 7.74

### Example 4

### Synthesis of Compound Represented by Chemical Formula (A-15)

A compound represented by the above Chemical Formula (A-15) as specific examples of a compound for an organic optoelectronic device of the present invention was synthesized according to the following Reaction Scheme 4.

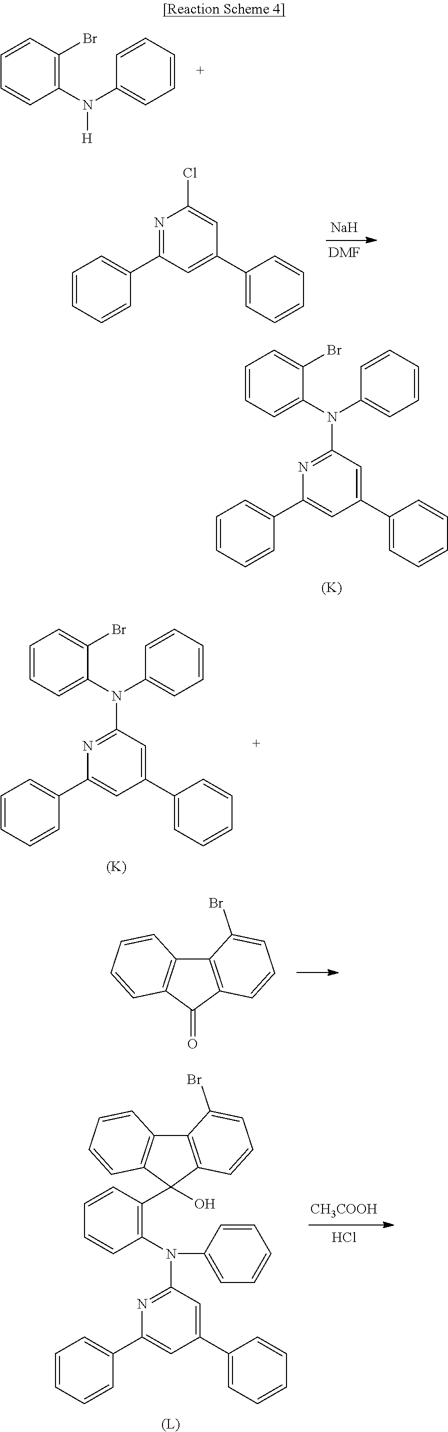

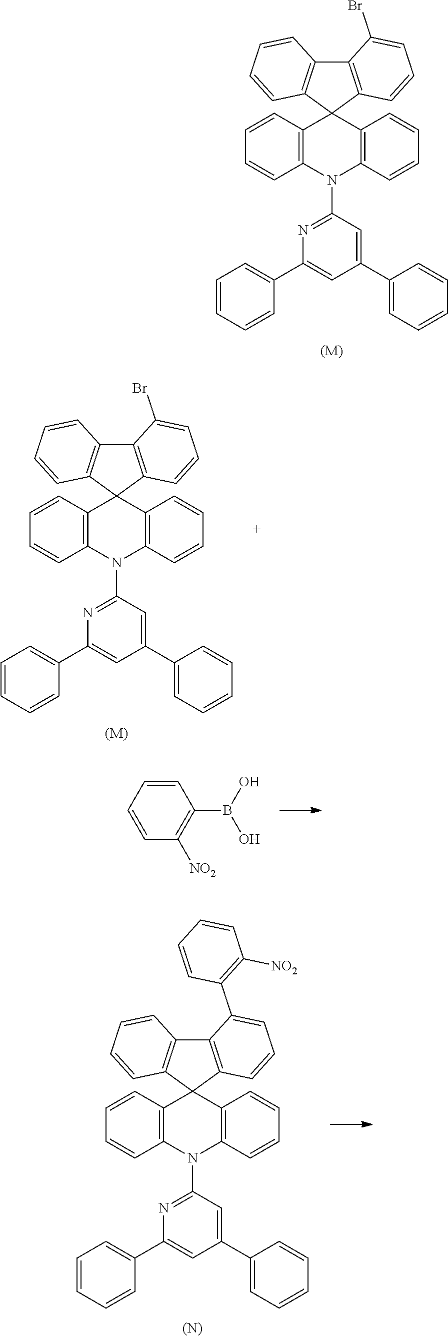

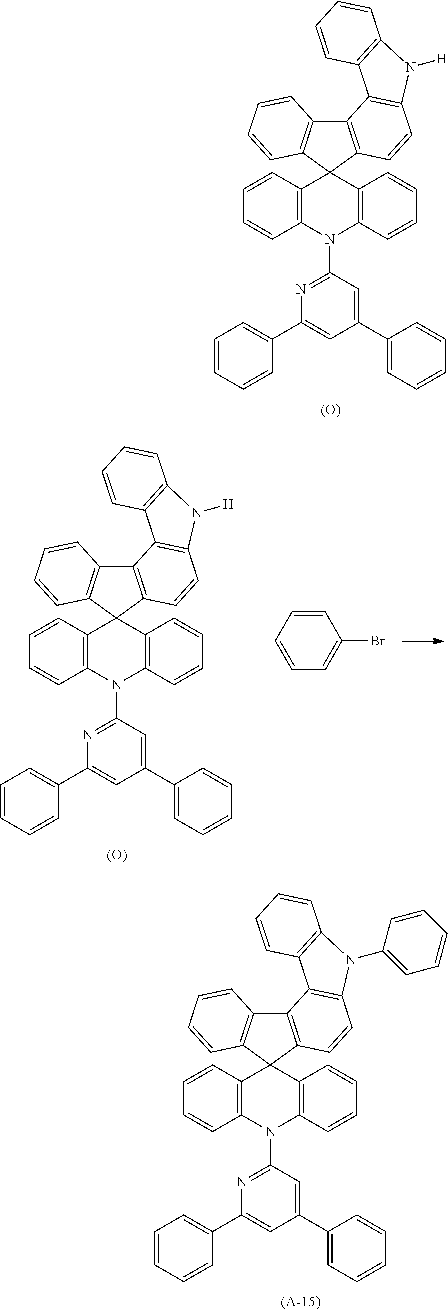

First Step; Synthesis of Compound (K)

30 g (120 mmol) of 2-bromo aniline, 38.3 g (150 mmol) of 1-chloro-3,5-phenyl pyridine, and 7.3 g (300 mmol) of sodium hydride were put in 900 ml of dimethyl formaldehyde in a 2000 mL round flask, and the mixture was agitated at room temperature for 24 hours. After removing non-reaction sodium hydride by slowly dropping the reaction solution to distilled water, the reaction solution was poured into an excessive water of distilled water, and the mixture was filtered. The obtained solid was dissolved in an excessive amount of methylene chloride, the solution was hot-filtered, methylene chloride was removed therefrom again, and a solid obtained by precipitating the resultant in methanol was filtered, obtaining 35.9 g of a compound (K) (a yield of 66%).

The elemental analysis result of the compound (K) was provided as follows.

calcd. C₂₉H₂₁BrN₂: C, 72.96; H, 4.43; Br, 16.74; N, 5.87. found: C, 72.94; H, 4.41; Br, 16.74; N, 5.88

Second step: Synthesis of Compound (L)

29.4 g (62.5 mmol) of the compound (K) was put in a 500 mL round flask and dissolved in 300 mL of tetrahydrofuran after making its reaction atmosphere be in a nitrogen state, and a temperature around a reactor was maintained at −78° C. Then, 25 mL of 2.5M-normal butyl lithium was slowly added thereto in a dropwise fashion. When the addition was complete, the mixture was agitated for 30 minutes, and a solution obtained by dissolving 16.2 g (62.5 mmol) of 4-bromo fluoreneol in 200 mL of purified tetrahydrofuran was slowly added thereto. The reaction solution was agitated for about one hour while maintained at −78° C. and then, heated up to room temperature and kept being agitated until the next day morning. After completing a reaction by adding a 5 wt % sodium bicarbonate aqueous solution to the reaction solution, the resultant was separately extracted by using methylene chloride. The obtained organic layer was distilled under a reduced pressure after removing water remaining there with magnesium sulfate, obtaining an intermediate (L). The unpurified intermediate (L) was put in 400 mL of acetic acid, hydrochloric acid in a catalyst amount was added thereto, and the mixture was agitated for 12 hours at a reflux temperature. When the reaction was complete, 27.3 g of a compound (M) was obtained through column chromatography (a yield of 64%).

The elemental analysis result of the compound (M) was provided as follows.

calcd. C₄₂H₂₇BrN₂: C, 78.87; H, 4.26; Br, 12.49; N, 4.38. found: C, 78.89; H, 4.25; N, 4.35

Third step: Synthesis of Compound (0)

19.4 g (31.1 mmol) of the compound (M), 6.3 g (31.1 mmol) of a 2-nitrobenzene boronic acid compound and 1.8 g (1.2 mmol) of tetrakis tetrakis triphenyl phosphine were suspended in 400 ml of toluene/tetrahydrofuran, 400 ml of a 2 M potassium carbonate aqueous solution was added thereto, and the mixture was heated and refluxed under a nitrogen stream for 24 hours. The reaction solution was added to 2000 ml of MeOH, a crystallized solid therein was filtered and dissolved in monochlorobenzene, and the solution was filtered with silica gel/Celite. After removing an organic solvent in an appropriate amount therefrom, The resultant was recrystallized in MeOH, obtaining compound (N) 14.2 g (yield 70%) [symbol] [symbol] .

14.2 g (20 mmol) of the compound (N) was agitated with 18 g (100 mmol) of triethyl phosphite under a nitrogen stream for one night. After removing untreated triethyl phosphite by a vacuum distillation method, 8.2 g (13.2 mmol) of a compound (O) was obtained through column chromatography for a solid remaining in the flask.

The elemental analysis result of the compound (O) was provided as follows.

calcd. C₄₈H₃₁N₃: C, 88.72; H, 4.81; N, 6.47. found: C, 88.73; H, 4.82; N, 6.56

Fourth Step; Synthesis of Compound (A-15)

8.2 g (13 mmol) of the compound (O), 2.5 g (15.6 mmol) of bromo benzene and 2.8 g (19.5 mmol) of potassium carbonate were in 250 ml of DMSO, 0.5 g (0.3 mmol) of 1,10-phenanthroline and 0.3 g (0.3 mmol) of copper chloride were added, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 5.2 g of the compound (A-15) (a yield of 60%).

The elemental analysis result of the compound (A-15) was provided as follows.

calcd. C₅₄H₃₅N₃: C, 89.35; H, 4.86; N, 5.79. found: C, 89.31; H, 4.84; N, 5.75

### Example 5

### Synthesis of Compound Represented by Chemical Formula (A-21)

A compound represented by the above Chemical Formula (A-21) as specific examples of a compound for an organic optoelectronic device according to the present invention was synthesized according to the following Reaction Scheme 5.

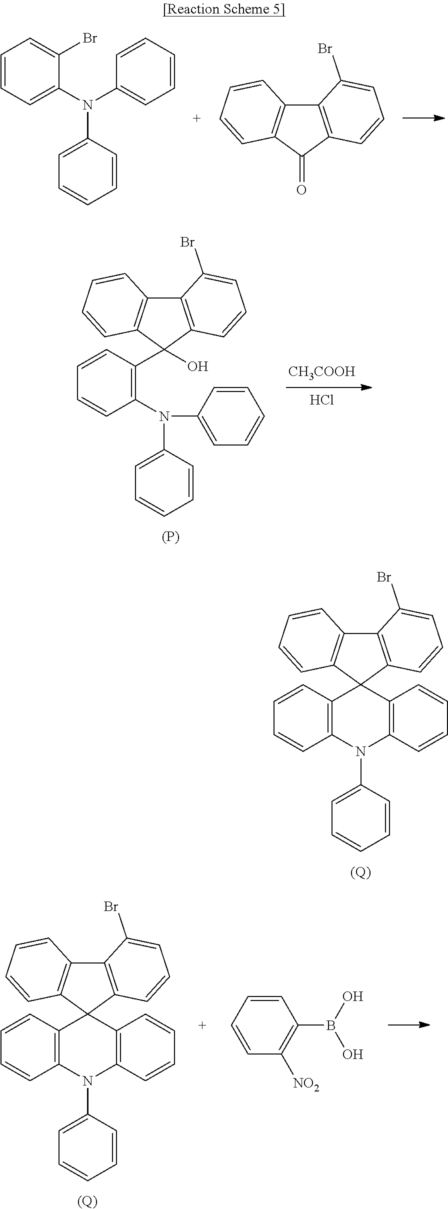

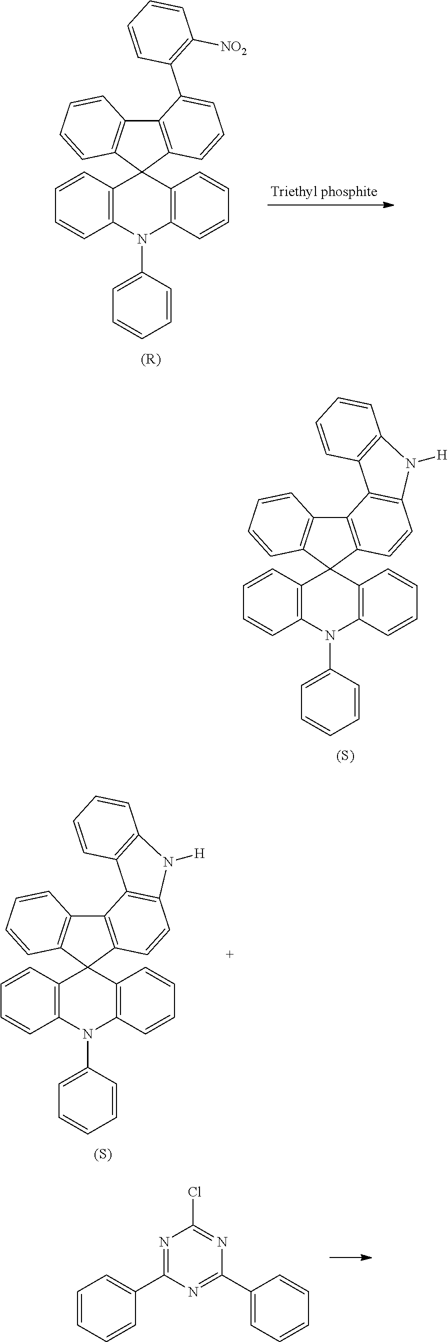

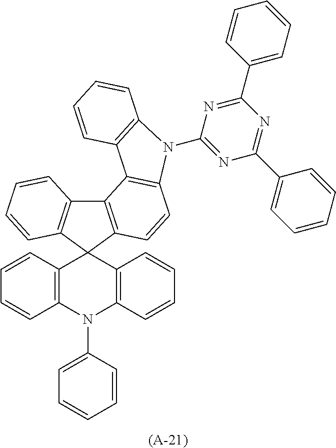 [SMILES: c1ccc(-c2nc(-c3ccccc3)nc(-n3c4ccccc4c4c5c(ccc43)C3(c4ccccc4-5)c4ccccc4N(c4ccccc4)c4ccccc43)n2)cc1 ; "(A-21)"]

First Step; Synthesis of Compound (Q)

20.3 g (62.5 mmol) of bromo triphenyl amine was put in a 500 mL round flask and dissolved in 300 mL of tetrahydrofuran after making its reaction atmosphere be in a nitrogen state, and a temperature around a reactor was maintained at −78° C. Then, 25 mL of 2.5M-normal butyl lithium was slowly added thereto in a dropwise fashion. When the addition was complete, the mixture was agitated for 30 minutes, and a solution obtained by dissolving 16.2 g (62.5 mmol) of 4-bromo fluoreneol in 200 mL of purified tetrahydrofuran was slowly added thereto. The reaction solution was agitated for about one hour while maintained at −78° C. and then, heated up to room temperature and kept being agitated until the next day morning. After completing a reaction by adding a 5 wt % sodium bicarbonate aqueous solution to the reaction solution, the resultant was separately extracted by using methylene chloride. The obtained organic layer was distilled under a reduced pressure after removing water remaining there with magnesium sulfate, obtaining an intermediate (P). The unpurified intermediate (P) was put in 400 mL of acetic acid, hydrochloric acid in a catalyst amount was added thereto, and the mixture was agitated for 12 hours at a reflux temperature. When the reaction was complete, 19.5 g of a compound (Q) was obtained through column chromatography (a yield of 64%).

The elemental analysis result of the compound (Q) was provided as follows.

calcd. C₃₁H₂₀BrN: C, 76.55; H, 4.14; Br, 16.43; N, 2.88. found: C, 76.51; H, 4.11; N, 2.85

Second Step; Synthesis of Compound (S)

15.1 g (31.1 mmol) of the compound (Q), 6.3 g (31.1 mmol) of a 2-nitrobenzene boronic acid compound and 1.8 g (1.2 mmol) of tetrakis tetrakis triphenyl phosphine were suspended in 400 ml of toluene/tetrahydrofuran, 400 ml of a 2 M potassium carbonate aqueous solution was added thereto, and the mixture was heated and refluxed under a nitrogen stream for 24 hours. The reaction solution was added to 2000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining compound (R) 11.5 g (yield 70%).

11.5 g (20 mmol) of the compound (R) was agitated with 18 g (100 mmol) of triethyl phosphite under a nitrogen stream for one night. After removing untreated triethyl phosphite by a vacuum distillation method, 6.5 g (13.2 mmol) of a compound (S) was obtained through column chromatography for a solid remaining in the flask.

The elemental analysis result of the compound (S) was provided as follows.

calcd. C₃₇H₂₄N₂: C, 89.49; H, 4.87; N, 5.64. found: C, 89.49; H, 4.89; N, 5.61

Third step: Synthesis of Compound (A-21)

6.5 g (13 mmol) of the compound (S), 4.2 g (15.6 mmol) of 1-chloro-3,5-phenyl triazine and 2.8 g (19.5 mmol) of potassium carbonate were suspended in 250 ml of DMSO, 0.5 g (0.3 mmol) of 1,10-phenanthroline and 0.3 g (0.3 mmol) of copper chloride were added thereto, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 5.7 g of a compound (A-21) (a yield of 60%).

The elemental analysis result of the compound (A-21) was provided as follows.

calcd. C₅₂H₃₃N₅: C, 85.81; H, 4.57; N, 9.62. found: C, 85.83; H, 4.52; N, 9.59

### Example 6

### Synthesis of Compound Represented by Chemical Formula (A-20)

A compound represented by the above Chemical Formula (A-20) as specific examples of a compound for an organic optoelectronic device according to the present invention was synthesized according to the following Reaction Scheme 6.

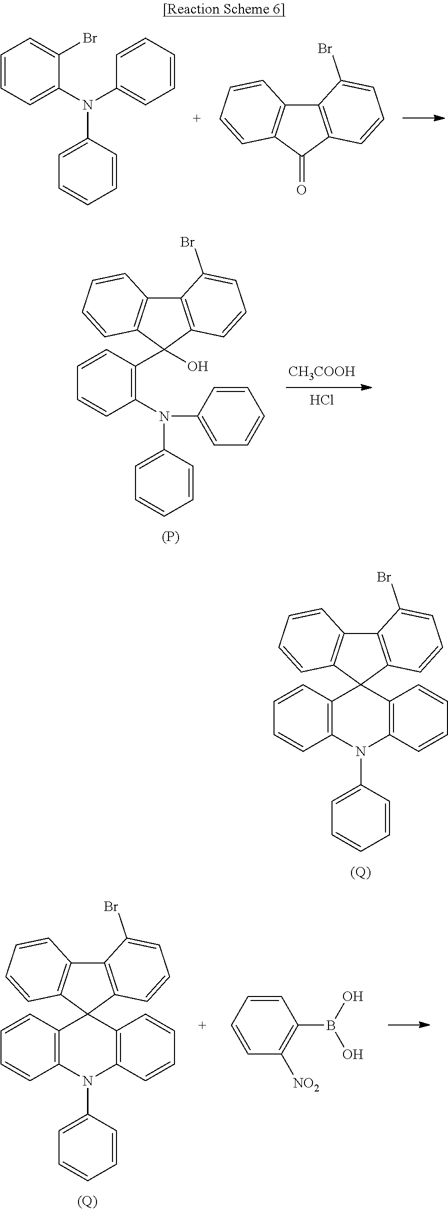

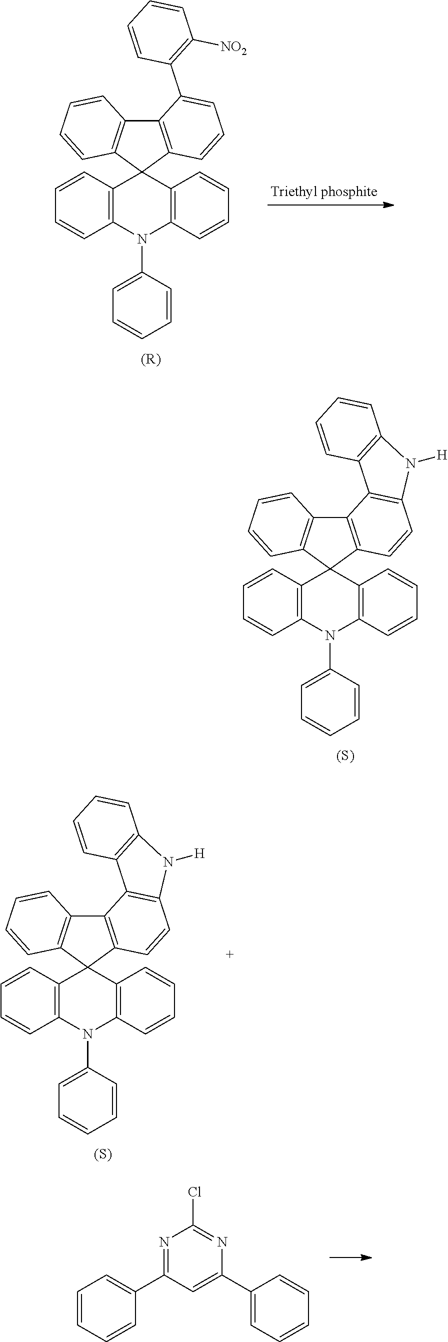

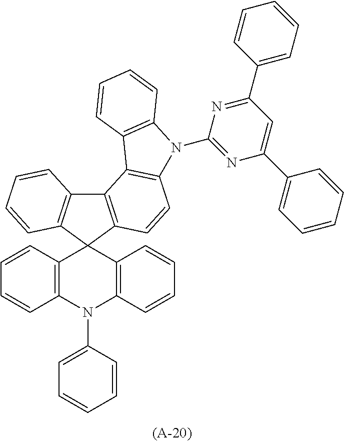 [SMILES: c1ccc(-c2cc(-c3ccccc3)nc(-n3c4ccccc4c4c5c(ccc43)C3(c4ccccc4-5)c4ccccc4N(c4ccccc4)c4ccccc43)n2)cc1 ; "(A-20)"]

First Step; Synthesis of Compound (A-20)

6.5 g (13 mmol) of the compound (S) synthesized through the first and second steps in Example 5, 4.2 g (15.6 mmol) of 1-chloro-3,5-phenyl pyrimidine and 2.8 g (19.5 mmol) of potassium carbonate were suspended in 250 ml of DMSO, 0.5 g (0.3 mmol) of 1,10-phenanthroline and 0.3 g (0.3 mmol) of copper chloride were added thereto, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 5.6 g of a compound (A-20) (a yield of 60%).

The elemental analysis result of the compound (A-20) was provided as follows.

calcd. Cₛ₃H₃₄N₄: C, 87.58; H, 4.71; N, 7.71. found: C, 87.55; H, 4.67; N, 7.74

### Example 75

### Synthesis of Compound Represented by Chemical Formula (A-19)

A compound represented by the above Chemical Formula (A-19) as specific examples of a compound for an organic optoelectronic device according to the present invention was synthesized according to the following Reaction Scheme 7.

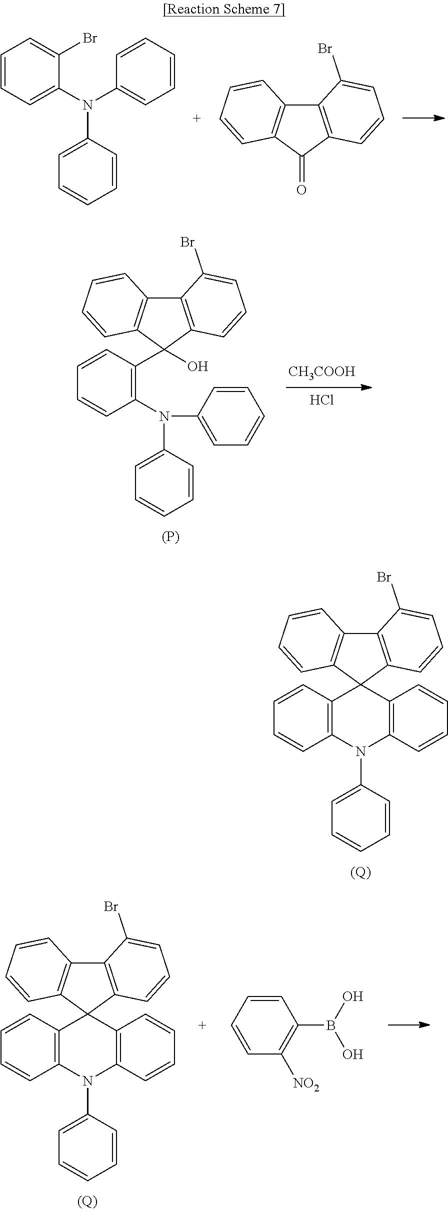

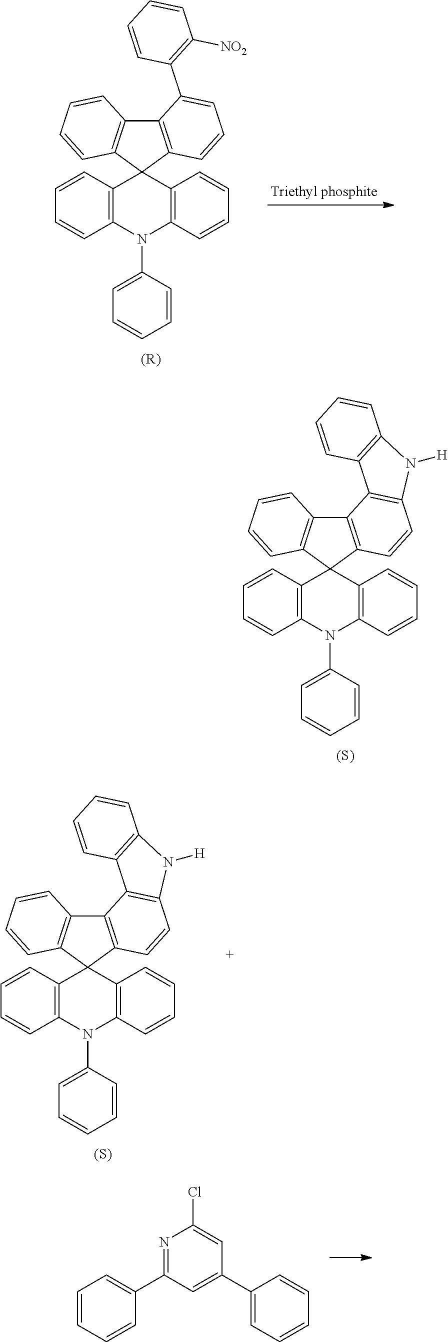

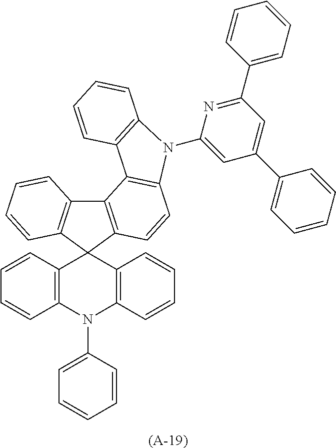 [SMILES: c1ccc(-c2cc(-c3ccccc3)nc(-n3c4ccccc4c4c5c(ccc43)C3(c4ccccc4-5)c4ccccc4N(c4ccccc4)c4ccccc43)c2)cc1 ; "(A-19)"]

First Step; Synthesis of Compound (A-19)

6.5 g (13 mmol) of the compound (S) synthesized through the first and second steps in Example 5, 4.1 g (15.6 mmol) of 1-chloro-3,5-phenyl pyridine and 2.8 g (19.5 mmol) of potassium carbonate were suspended in 250 ml of DMSO, 0.5 g (0.3 mmol) of 1,10-phenanthroline and 0.3 g (0.3 mmol) of copper chloride were added thereto, and the mixture was heated and refluxed under a nitrogen stream for 12 hours. The reaction solution was added to 1000 ml of MeOH, a crystallized solid therein was filtered and dissolved in monochlorobenzene, and the solution was filtered with silica gel/Celite. After removing an organic solvent in an appropriate amount therefrom, the resultant was recrystallized in MeOH, obtaining 5.5 g of a compound (A-19) (a yield of 60%).

The elemental analysis result of the compound (A-19) was provided as follows.

calcd. C₅₄H₃₅N₃: C, 89.35; H, 4.86; N, 5.79. found: C, 89.37; H, 4.83; N, 5.82

(Manufacture of Organic Light Emitting Diode)

### Example 8

Specifically, illustrating a method of manufacturing an organic light emitting diode, an anode was manufactured by cutting an ITO glass substrate having a sheet resistance of 15 Ω/cm² into a size of 50 mm×50 mm×0.7 mm, respectively ultrasonic wave-cleaning it in acetone, isopropylalcohol and pure water for 15 minutes and then, UV ozone-cleaning it for 30 minutes.

This obtained ITO transparent electrode was used as an anode, and a 1200 Å-thick hole injection layer (HIL) was formed on the ITO substrate by vacuum-depositing the following HTM compound.

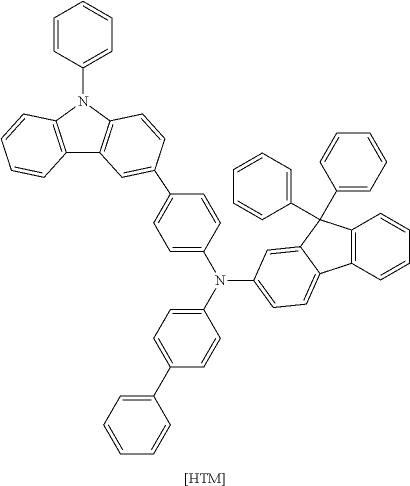

Then, the compound synthesized in Example 1 as a host and 7 wt % of the following PhGD compound as a phosphorescent green dopant were vacuum-deposited to form a 300 Å-thick emission layer. As for an anode, 1000 Å-thick aluminum (Al) was used, while 1000 Å-thick ITO was used as for a cathode.

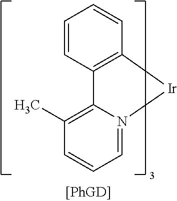

Then, on the emission layer, 50 Å-thick BAlq [bis(2-methyl-8-quinolinolato-N1,O8)-(1,1′-Biphenyl-4-olato)aluminum] and 250 Å-thick Alq3 [tris(8-hydroxyquinolinato)aluminium] were sequentially accumulated to form an electron transport layer (ETL). On the electron transport layer (ETL), 5 Å-thick LiF and 1000 Å-thick Al were sequentially vacuum-deposit to form a cathode, manufacturing an organic light emitting diode.

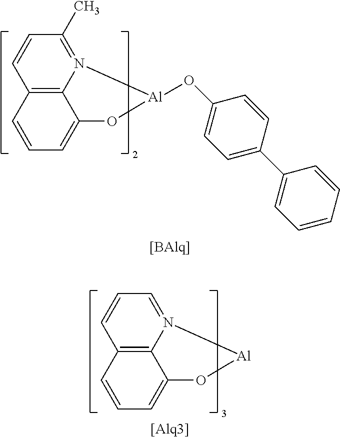

### Example 9

An organic light emitting diode was manufactured according to the same method as Example 8 except for using the compound of Example 5 instead of the compound of Example 1 in Example 9.

### Comparative Example 1

An organic light emitting diode was manufactured according to the same method as Example 8 except for using 4,4-N,N-dicarbazolebiphenyl (CBP) instead of the compound synthesized in Example 1 as the host for the emission layer in Example 9.

(Performance Measurement of Organic Light Emitting Diode)

Current density change, luminance change, and luminous efficiency of each organic light emitting diode according to the Examples 9 and 10 and Comparative Example 1 depending on a voltage were measured. Specific measurement methods are as follows, and the results are shown in the following Table 1.

(1) Measurement of Current Density Change Depending on Voltage Change

The obtained organic light emitting diodes were measured for current value flowing in the unit device while increasing the voltage from 0 V to 10 V using a current-voltage meter (Keithley 2400), the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

The luminance, current density, and voltage obtained from the (1) and (2) were used to calculate current efficiency (cd/A) at the same current density (10 mA/cm²).

TABLE 1

| | Driving | Current | Power | | Color | Color |
|---|---|---|---|---|---|---|
| | voltage | efficiency | efficiency | Luminance | coordinate | coordinate |
| | (Vd, V) | (cd/A) | (lm/W) | (cd/m²) | (CIEx) | (CIEy) |
| Comparative | 4.05 | 37.1 | 34.1 | 3000 | 0.339 | 0.625 |
| Example 1 | | | | | | |
| Example 8 | 4.12 | 39.1 | 38.4 | 3000 | 0.351 | 0.617 |
| Example 9 | 4.16 | 38.6 | 38.2 | 3000 | 0.348 | 0.620 |

The organic light emitting diodes of Examples 8 to 9 showed improved efficiency compared with the organic light emitting diodes of Comparative Example 1 using CBP as the host for the emission layer.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by the following Chemical Formula 1: 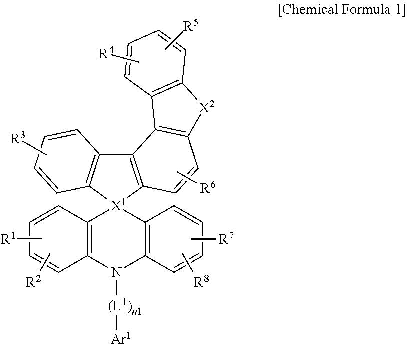
wherein, in Chemical Formula 1,
X¹ is C or Si,
X² is O, S, SO₂ (O═S═O), PO (P═O), CR′R″, or NR′,
R′, R″, and R¹ to R⁸ are each independently hydrogen, deuterium, a halogen, a cyano group, a hydroxyl group, an amino group, a substituted or unsubstituted C1 to C20 amine group, a nitro group, a carboxyl group, a ferrocenyl group, a substituted or unsubstituted C1 to C20 alkyl group, a substituted or unsubstituted C6 to C30 aryl group, a substituted or unsubstituted C2 to C30 heteroaryl group, a substituted or unsubstituted C1 to C20 alkoxy group, a substituted or unsubstituted C6 to C20 aryloxy group, a substituted or unsubstituted C3 to C40 silyloxy group, a substituted or unsubstituted C1 to C20 acyl group, a substituted or unsubstituted C2 to C20 alkoxycarbonyl group, a substituted or unsubstituted C2 to C20 acyloxy group, a substituted or unsubstituted C2 to C20 acylamino group, a substituted or unsubstituted C2 to C20 alkoxycarbonylamino group, a substituted or unsubstituted C7 to C20 aryloxycarbonylamino group, a substituted or unsubstituted C1 to C20 sulfamoylamino group, a substituted or unsubstituted C1 to C20 sulfonyl group, a substituted or unsubstituted C1 to C20 alkylthiol group, a substituted or unsubstituted C6 to C20 arylthiol group, a substituted or unsubstituted C1 to C20 heterocyclothiol group, a substituted or unsubstituted C1 to C20 ureide group, a substituted or unsubstituted C3 to C40 silyl group, or a combination thereof,
L′ is a substituted or unsubstituted C2 to C6 alkenylene group, a substituted or unsubstituted C2 to C6 alkynylene group, a substituted or unsubstituted C6 to C30 arylene group, a substituted or unsubstituted C2 to C30 heteroarylene group, or a combination thereof,
n1 is an integer of 0 to 3,
Ar¹ is a substituted or unsubstituted C6 to C30 aryl group or a substituted or unsubstituted C2 to C30 heteroaryl group, and
at least one of Ar¹, R⁸, and R′ is a substituted or unsubstituted C2 to C30 heteroaryl group having electron characteristics.

2. The compound for an organic optoelectronic device as claimed in claim 1, wherein:
X² is O, S, or NR′, and
Ar¹ is a substituted or unsubstituted C2 to C30 heteroaryl group having electron characteristics.

3. The compound for an organic optoelectronic device as claimed in claim 2, wherein:
X² is NR, and
R′ is a substituted or unsubstituted C6 to C30 aryl group.

4. The compound for an organic optoelectronic device as claimed in claim 1, wherein:
X² is NR′, and
R′ is substituted or unsubstituted C2 to C30 heteroaryl group having electron characteristics.

5. The compound for an organic optoelectronic device as claimed in claim 4, wherein Ar¹ is a substituted or unsubstituted C6 to C30 aryl group.

6. The compound for an organic optoelectronic device as claimed in claim 1, wherein the compound represented by Chemical Formula 1 is represented by the following Chemical Formula 2: 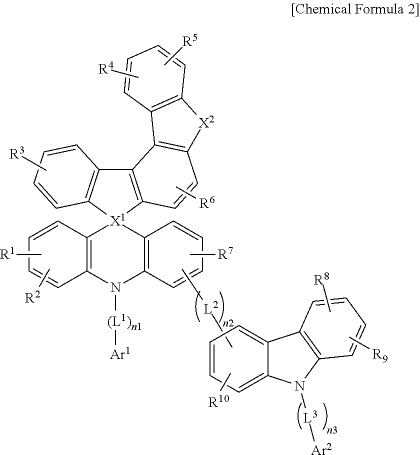
wherein, in Chemical Formula 2,
X¹ is C or Si,
X² is O, S, SO₂ (O═S═O), PO (P═O), CR′R″ or NR′,
R, R″, and R¹ to R¹⁰ are each independently hydrogen, deuterium, a halogen, a cyano group, a hydroxyl group, an amino group, a substituted or unsubstituted C1 to C20 amine group, a nitro group, a carboxyl group, a ferrocenyl group, a substituted or unsubstituted C1 to C20 alkyl group, a substituted or unsubstituted C6 to C30 aryl group, a substituted or unsubstituted C2 to C30 heteroaryl group, a substituted or unsubstituted C1 to C20 alkoxy group, a substituted or unsubstituted C6 to C20 aryloxy group, a substituted or unsubstituted C3 to C40 silyloxy group, a substituted or unsubstituted C1 to C20 acyl group, a substituted or unsubstituted C2 to C20 alkoxycarbonyl group, a substituted or unsubstituted C2 to C20 acyloxy group, a substituted or unsubstituted C2 to C20 acylamino group, a substituted or unsubstituted C2 to C20 alkoxycarbonylamino group, a substituted or unsubstituted C7 to C20 aryloxycarbonylamino group, a substituted or unsubstituted C1 to C20 sulfamoylamino group, a substituted or unsubstituted C1 to C20 sulfonyl group, a substituted or unsubstituted C1 to C20 alkylthiol group, a substituted or unsubstituted C6 to C20 arylthiol group, a substituted or unsubstituted C1 to C20 heterocyclothiol group, a substituted or unsubstituted C1 to C20 ureide group, a substituted or unsubstituted C3 to C40 silyl group, or a combination thereof,
L¹ to L³ are each independently a substituted or unsubstituted C2 to C6 alkenylene group, a substituted or unsubstituted C2 to C6 alkynylene group, a substituted or unsubstituted C6 to C30 arylene group, a substituted or unsubstituted C2 to C30 heteroarylene group, or a combination thereof,
n1 to n3 are independently integers of 0 to 3,
Ar¹ and Ar² are each independently a substituted or unsubstituted C6 to C30 aryl group or a substituted or unsubstituted C2 to C30 heteroaryl group, and
at least one of Ar¹, Ar², R⁵, R⁸, and R′ is a substituted or unsubstituted C2 to C30 heteroaryl group having electron characteristics.

7. The compound for an organic optoelectronic device as claimed in claim 6, wherein:
X² is O, S, or CR′R″, and
Ar¹ is a substituted or unsubstituted C2 to C30 heteroaryl group having electron characteristics.

8. The compound for an organic optoelectronic device as claimed in claim 7, wherein:
X² is O or S, and
Ar² is a substituted or unsubstituted C6 to C30 aryl group.

9. The compound for an organic optoelectronic device as claimed in claim 6, wherein:
X² is O or S,
Ar² is a substituted or unsubstituted C2 to C30 heteroaryl group having electron characteristics, and
Ar¹ is a substituted or unsubstituted C6 to C30 aryl group.

10. The compound for an organic optoelectronic device as claimed in claim 6, wherein X¹ is C.

11. The compound for an organic optoelectronic device as claimed in claim 1, wherein the substituted or unsubstituted C2 to C30 heteroaryl group having electron characteristics is a group represented by one of the following Chemical Formulae 3 to 7: 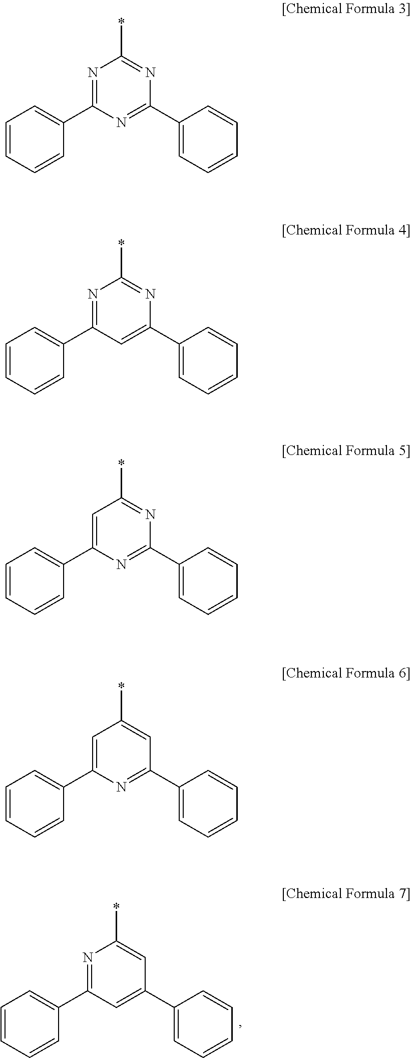 [SMILES: *c1nc(-c2ccccc2)nc(-c2ccccc2)n1 ; *c1nc(-c2ccccc2)cc(-c2ccccc2)n1 ; *c1cc(-c2ccccc2)nc(-c2ccccc2)n1 ; *c1cc(-c2ccccc2)nc(-c2ccccc2)c1 ; *c1cc(-c2ccccc2)cc(-c2ccccc2)n1]
in which * represents a binding site to a neighboring atom.

12. The compound for an organic optoelectronic device as claimed in claim 6, wherein Ar¹ and Ar² are each independently a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenylyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, or a combination thereof.

13. The compound for an organic optoelectronic device as claimed in claim 6, wherein L¹ to L³ are each independently a substituted or unsubstituted phenylene group, a substituted or unsubstituted biphenylene group, a substituted or unsubstituted terphenylene group, a substituted or unsubstituted naphthylene group, a substituted or unsubstituted anthracenylene group, a substituted or unsubstituted phenanthrylene group, a substituted or unsubstituted pyrenylene group, a substituted or unsubstituted fluorenylene group, a substituted or unsubstituted naphthacenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group and a substituted or unsubstituted phenoxazinyl group.

14. The compound for an organic optoelectronic device as claimed in claim 6, wherein at least one of R¹ toe is a substituted or unsubstituted C3 to C40 silyl group.

15. The compound for an organic optoelectronic device as claimed in claim 6, wherein at least one of R¹ to R¹⁰ is a substituted C3 to C40 silyl group, the substituted C3 to C40 silyl being substituted with at least one of a C1 to C10 alkyl group or a C6 to C15 aryl group.

16. The compound for an organic optoelectronic device as claimed in claim 1, wherein the compound has a triplet exciton energy of 2.0 eV or greater.

17. An organic light emitting diode, comprising:
an anode;
a cathode; and
at least one organic thin layer between the anode and the cathode,
wherein the at least one organic thin layer includes the compound for an organic optoelectronic device as claimed in claim 1.

18. The organic light emitting diode as claimed in claim 17, wherein the at least one organic thin layer includes an emission layer, a hole transport layer (HTL), a hole injection layer (HIL), an electron transport layer (ETL), an electron injection layer (EIL), a hole blocking layer, or a combination thereof.

19. The organic light emitting diode as claimed in claim 17, wherein:
the at least one organic thin layer includes an emission layer, and
the compound for an organic optoelectronic device is included in the emission layer.

20. A display device comprising the organic light emitting diode as claimed in claim 17.
